(19) United States
(12) Patent Application Publication
(10) Pub. No.: US 2004/0028656 A1
(43) Pub. Date: Feb. 12, 2004

(54) Survival of neurons

(75) Inventors: Allison E. Willing, Tampa, FL (US); Tanya Zigova, Tampa, FL (US); Paul R. Sanberg, Spring Hill, FL (US); Michael McGrogan, San Carlos, CA (US); Gary Snable, Atherton, CA (US)

Correspondence Address: QUARLES & BRADY LLP, RENAISSANCE ONE, TWO NORTH CENTRAL AVENUE, PHOENIX, AZ, 85004-2391, US

(73) Assignees: University of South Florida, a non-profit institution; Layton Bioscience, Inc.

(21) Appl. No.: 10/313,915

(22) Filed: Dec. 6, 2002

Related U.S. Application Data
(63) Continuation-in-part of application No. 09/494,088, filed on Jan. 28, 2000, now abandoned.
Application No. 09/494,088 is a continuation-in-part of application No. PCT/US98/23977, filed on Nov. 10, 1998.
(60) Provisional application No. 60/094,515, filed on Jul. 29, 1998.

(51) Int. Cl. A01N 1/02; C12N 5/08

(52) U.S. Cl. 424/93.7; 435/2; 435/368

(57) ABSTRACT

A method of improving the survival of neuronal cells has the steps of obtaining a frozen cellular composition comprising neuronal cells; thawing the cellular composition; and contacting the cellular composition with a balanced electrolyte solution including a lithium salt. One example of a lithium salt is lithium chloride. Also is provided a kit comprising a container of with a cellular composition comprising neuronal cells and a container of a diluent comprising a balanced electrolyte solution and a lithium salt.

### CROSS-REFERENCE TO RELATED APPLICATIONS

This application is a continuation-in-part of pending application Ser. No. 09/494,088, filed Jan. 28, 2000, which is a continuation-in-part of International Application serial no. PCT/US98/23977 filed Nov. 10, 1998, which claims benefit of provisional application serial No. 60/094,515 filed Jul. 29, 1998.

### BACKGROUND

1. Technical Field

The present invention is in the field of tissue cell culturing and relates more particularly to methods for increasing the survival of neurons and increasing the numbers of dopaminergic cells by treatment with a lithium salt.

2. The Prior Art

Dopaminergic neurons are those that synthesize and use dopamine (DA) as a neurotransmitter. Dopaminergic neurons are found in a number of areas of the brain, including the nigrostriatal, mesolimbic, mesocortical and tubero-hypophysial systems. The rate-limiting step in dopamine synthesis is catalysis of tyrosine by tyrosine hydroxylase (TH). Dopamine is stored in pre synaptic vesicles and released From there by exocytosis. Dopamine acts on as many as five classes of receptors. Dopamine is recycled by reuptake and/or degradation by monoamine oxidase B (MAO-B) (R K Murray, Ch. 64. The Biochemical Basis of Some Neuropsychiatric Disorders. In: Harper's Biochemistry, ed. by Murray, et al. 24^{th} ed., Appleton & Lange, Stamford, Conn., 1996, pp. 794-814).

Parkinson's disease (PD) is a neurodegenerative disorder characterized by a loss of dopaminergic cells from the substantia nigra par compacta, resulting in decreased dopaminergic input to the striatum. The hallmark motor symptoms include tremor, rigidity, bradykinesia, and instability. In spite of a host of approved pharmacological and surgical treatments, existing therapies for PD are only partial and palliative. Levodopa (L-dopa) the gold standard pharmacological treatment to restore DA, is plagued by decreased efficacy and increased side effects over time. Adjunct treatment with DA agonists is frequently necessary; however, recently approved DA agonists with greater receptor subtype specificity may provide only incremental clinical benefit. Catechol-O-methyltransferase (COMT) inhibitors to slow DA metabolism soon will be joining monoamine oxidase (MAO) inhibitors.

To replace the missing cells, there has been a renaissance of neurosurgical treatments for PD. After all pharmacological treatments have failed, surgical procedures including pallidotomy, thalamotomy and deep electrical stimulation may be considered. Nevertheless, for almost one million individuals in the US afflicted by PD, a reliable long-term treatment to halt disease progression remains elusive.

Schizophrenia is often treated by neuroleptic drugs which decrease the amount of dopamine activity in mesolimbic dopaminergic neurons. “Positive symptoms” (e.g., hallucinations, delusions, bizarre behavior) have been associated with excess dopamine activity in the mesolimbic neurons. “Negative symptoms” of schizophrenia (e.g., social withdrawal, emotional blunting, and catatonia) may be associated with low dopamine activity in the prefrontal cortex. Since prefrontal dopaminergic neurons may normally inhibit the activity of subcortical dopamine neurons, a lowering of dopamine in the prefrontal area could lead to the elevated dopaminergic activity in the subcortical neurons.

Progressive Supranuclear Palsy (Steele-Richardson-Olszewski Syndrome) is due to a loss of neurons and gliosis in the tectum and tegmentum of the midbrain, the subthalamic nuclei of Luys, the vestibular nuclei, and to some extent the ocular nuclei. Some symptoms are shared with Parkinson's disease, including rigidity of the neck and other trunk muscles and occasional sensitivity to L-dopa.

A rare form of torsion dystonia is dramatically L-dopa-responsive. Starting in childhood, the dystonia first affects gait. Most individuals later develop parkinsonism. Some focal dystonias also are reported to be L-dopa responsive.

In a neurodegenerative disorder associated with autonomic failure (i.e., Shy-Drager Syndrome), positron emission tomography has shown decreased uptake of dopamine derivatives in the putamen and caudate, probably reflecting a loss of nigrostriatal dopaminergic neurons. Current treatment is symptomatic. The parkinsonian symptoms may be helped by L-dopa or other dopaminergic drugs, but later most patients become refractory to these drugs.

Depression is associated with heterogeneous dysregulation of the biogenic amines. Although norepinephrine and serotonin have been most implicated in the pathophysiology, dopamine also may play a role in depression. Dopamine may be reduced in depression and increased in mania. Drugs that reduce dopamine concentrations (e.g., reserpine) and diseases that reduce dopamine concentrations (e.g., Parkinson's disease) are associated with depressive symptoms. Also, drugs that increase dopamine concentrations (e.g., tyrosine, amphetamine and bupropion) reduce the symptoms of depression. Two recent theories regarding dopamine and depression are that the mesolimbic dopamine pathway may be dysfunctional in depression and that the dopamine type 1 (D1) receptor may be hypoactive in depression (Ch.9. Mood Disorders, in: CONCISE TEXTBOOK OF CLINICAL PSYCHIATRY. Ed. by H I Kaplan and B J Sadock. Williams & Wilkins, Baltimore, Md., 1996, pp. 159-188).

MAO inhibitors also are the drugs of choice in agoraphobia (irrational fear of being alone or in public places) and panic disorder. There also is growing evidence that MAO inhibitors are effective in the treatment of some anxiety disorders, particularly mixed depressed and anxious states.

The search for a continuous, stable, regulated, site-specific source of DA delivery has turned to tissue transplantation, cell therapy and genetic engineering, with the ultimate goal of finding an effective treatment to halt or reverse disease progression.

Human fetal mesencephalic tissue transplants have been extensively studied. They have demonstrated therapeutic potential in animal models of PD and in Parkinson's disease patients. Fetal tissue transplants have been performed in the clinic for over a decade on more than 200 patients throughout the world with positive outcomes (Kordower J H, Goetz C G, Freeman T B, Olanow C W. Experimental Neurology 144:41-46, 1997). Grafts survive, form synaptic connections, and improve motor function in many patients. However, ethical, moral and technical constraints limit the widespread use of human fetal tissue.

Xenotransplantation, the use of cells from different species, is a viable approach to circumventing the limitations associated with human fetal neural transplantation (Galpern W R, Burns L H, Deacon T W, Dinsmore J, Isacson O. Experimental Neurology 140:1-13, 1996). Transplants of porcine cells harvested from the midbrains of pig fetuses have been evaluated. Another technique, developed by Cytotherapeutics, Inc., uses encapsulated xenografts of rat PC12 cells that secrete dopamine. Although cells derived from animals are potential candidates for human neural transplantation, they carry the risks of transferring intrinsic pathogens, creating novel infectious agents, or eliciting deleterious immune responses (Isacson O, Breakefield X. Nature Medicine 3:964-969, 1997).

Cell therapy for PD has the potential of reversing neurotransmitter deficiencies, halting neural degeneration, and repairing neural damage. Many types of cells (e.g., rat fibroblasts) have successfully been transfected ex vivo with, for example, the human tyrosine hydroxylase (TH) gene to generate dopaminergic factors locally (Raymon H K, Thode S, Gage F H. Experimental Neurology 144:82-91, 1997). Concerns about long-term stable gene expression, tumor formation, and pathogen delivery still need to be resolved.

In vivo gene therapy is possible by direct insertion of genes into brain cells via viral vectors (e.g., herpes simplex virus, adenovirus, adeno-associated virus, or lentivirus). Vectors encoding genes such as TH or glial-derived neurotrophic factor have been genetically engineered into cell lines. However, transplantation of genetically engineered cells into animal models of PD has not provided conclusive, long-term benefits or reinnervated the dopamine-depleted striatum. Moreover, the extent of gene expression, long-term efficacy, and cytopathogenicity associated with viral vectors is unknown.

Growth factors such as GDNF and brain-derived neurotrophic factor (BDNF) can be delivered alone or in combination with tissue transplants to provide trophic support and protect dopaminergic cells (Rosenblad C, Matinez-Serrano, Bjorklund A. Neuroscience 75:979-985, 1996). The long-term benefits and risks are unknown. Delivery is problematic, but novel approaches via injection directly into the brain, a Medtronic device, encapsulated cells, and genetically engineered cells are under investigation.

Recent research has focused on adapting NT2 or hNT cells for treatment of Parkinson's Disease (lacovitti and Stull, NeuroReport 8:1471-74, 1997). Both newly differentiating human neurons (hNT cells) and the undifferentiated precursors (NT2 cells) were treated with a variety of factors. In hNT neuronal cells but not NT2 precursor cells, TH expression was only induced by a combination of aFGF and co-activators (DA, TPA, or IBMX/forskolin), not individual factors. With increasing time in culture, more hNT cells expressed TH. After five days, 565 out of 10⁵ plated hNT cells, or less than 1%, expressed TH.

Other neuronal cells (i.e., hNT cells) have been implanted in humans who have experienced stroke and some clinical improvement has been reported, as have PET scans.

Lithium, a primary treatment for mania and bipolar affective disorder, has been reported to significantly influence the activity of signaling systems. Using PC12 cells as a model system, Li and Jope (J Neurochem 65:2500-08, 1995) studied the NGF-induced expression of several signal transduction proteins, including subtypes of G proteins, protein kinase C and phospholipase C and its modulation by lithium. Their results demonstrated that lithium, at a therapeutic concentration (1 mM), modulates the level of signal transduction proteins. Several studies have indicated that the activation of TH by intracellular calcium ion could be mediated by calcium/calmodulin-dependent protein kinase (for review, see Masserano et al., “The Role of TH in the Regulation of Catecholamine Synthesis.” In handbook of experimental pharmacology. Vol 90/II Catecholamines, Ed. by Trendelenburg and Weiner, Springer Verlag, Berlin, 1990, pp 427-69). However, controversial results have been obtained when lithium has been studied in relation to the brain content of catecholamines. Both decreased synthesis of dopamine (Friedman and Gershon, Nature 243:520-21, 1973) and up-regulated TH activity (Segal et al., Nature 254:58-59, 1975) have been reported after lithium treatment, perhaps due to the complexity of the brain tissue. On the other hand, increased synthesis and secretion of catecholamines and protein kinase C activity was demonstrated (Terao et al., Biol Psychiatry 31:1038-49, 1992) when lithium was applied on cultured adrenal medullary cells.

In summary, there is substantial evidence in both animal models and human patients that neural transplantation is a scientifically feasible and clinically promising approach to the treatment of PD. Nevertheless, alternative cell sources and novel strategies are needed to circumvent the numerous ethical and technical constraints that now limit the widespread use of neural transplantation.

According to Anton et al. (Anton R, et al. Exp Neurol 127:207-218, 1994), the ideal cell for a CNS transplant system should meet the following criteria: It should be of human CNS origin, capable of growth cessation and differentiation, clonal and defined, transfectable and selectable, immunologically inert, capable of long-term survival following implantation, non-tumorigenic, functional and integrated into the host brain, of consistent quality, and readily available.

### SUMMARY OF THE INVENTION

It is an object of the instant invention to provide neuronal cells that have an improved survival after transplantation.

A method of improving the survival of neuronal cells has the steps of obtaining a frozen cellular composition comprising neuronal cells; thawing the cellular composition; and contacting the cellular composition with a balanced electrolyte solution of a lithium salt. The lithium salt may be present in the range of about 0.25 mM to about 5 mM. The lithium salt may be in the range of about 0.5 mM to about 3 mM. The lithium salt may be in the range of about 0.75 mM to about 2 mM. The lithium salt concentration may be about 1 mM. The lithium salt can be lithium chloride.

In another embodiment, the method further comprises the step of centrifuging the thawed cellular composition and removing a resulting supernatant.

In another embodiment the viability of a portion of the neuronal cells in a balanced electrolyte solution is assessed.

In yet another embodiment, there is provided a kit comprising a container with a cellular composition comprising neuronal cells; and a container of a diluent comprising a balanced electrolyte solution and a lithium salt. The diluent's lithium salt concentration is greater than about 0.25 mM and less than about 5 mM. The diluent's lithium salt concentration is about 0.5 mM to about 3 mM, or about 0.75 mM to about 2 mM.

In yet another embodiment, there is provided a method of increasing the numbers of dopaminergic cells among neuronal cells. The method comprises providing a cellular composition comprising neuronal cells; and contacting the composition with a balanced electrolyte solution of a lithium salt for less than about 4 hours, thereby increasing the numbers of dopaminergic cells in the composition. The lithium salt may be present in the range of about 0.25 mM to about 5 mM, in the range of about 0.5 mM to about 3 mM, or in the range of about 0.75 mM to about 2 mM. The lithium salt can be lithium chloride.

In another embodiment, the cellular composition is centrifuged and a resulting supernatant is removed.

In another embodiment, the viability of a portion of the centrifuged neuronal cells is assessed.

### BRIEF DESCRIPTION OF THE DRAWING FIGURES

FIG. 1 is a tyrosine hydroxylase (TH) Western blot comparing different maturation conditions for the hNT neurons. It was developed with anti-TH monoclonal antibody and biotin-streptavidin alkaline phosphatase system. NT2/D1 cells were induced with retinoic acid (RetA) for 6 weeks and processed as Replate-I or Replate-II cultures in mitotic inhibitors for 1 week. Then the cultures were allowed to mature in conditioned media for 1 day (1 week replate), 1 week (2 week replate), or 2 weeks (3 weeks replate). Pure hNT neurons were harvested from the mature replate cultures and cell extracts corresponding to 1×10⁶ cells were loaded in the following lanes. Lanes 1-3 show the results for the Extended Replate-I Neurons which were matured for 1 week (lane 1), 2 weeks (lane 2), and 3 weeks (lane 3). Lanes 5 and 6 have Replate-II Neurons which were matured for 1 week and 2 weeks, respectively. Lanes 4 and 7 contain 1×10⁶ hNT neurons as positive controls.

FIG. 2 is a TH western blot showing the time course of RetA induction. It was developed with anti-TH monoclonal antibody and biotin-streptavidin alkaline phosphatase system. The NT2/D1 cells were induced with RetA for 4, 5, or 6 weeks, and after induction Replate-I cultures were maintained in mitotic inhibitors for either 1 week (Lanes 2-4) or a total of 2 weeks matured (Lanes 5-7). Purified neurons were harvested from each sample and cell extracts corresponding to 1×10⁶ cells were loaded in the following lanes: for the 1 week Replate-I: 2) 4w-RetA, 3) 5w-RetA, 4) 6w-RetA; and for the 2 weeks matured Replate I: 5) 4w-RetA, 6) 5w-RetA, 7) 6w-RetA, 8) hNT positive control; and rat TH standard was in lanes 1 (500 pg) and 9 (5ng).

FIGS. 3A-3G are photomicrographs of cultured neurons. DA neurons (3A and 3B) were immunostained for TH (arrows). For FIGS. 3A and 3B, the bar is 50 μm. FIG. 3C is a fluorescent photomicrograph showing TH+ hNT neurons (bar is 15 μm). FIGS. 3D and 3E are representative light photomicrographs of DAT-labeled DA neurons cultured an additional day and 5 days, respectively (bar is 50 μm). FIG. 3F shows hNT cells immunolabeled for DAT (bar is 25 μm). FIG. 3G is a fluorescent photomicrograph showing a clump of DA neurons (white asterisks) labeled with TH (green) and DAT (red) (bar is 10 μm).

FIGS. 4A and 4B are fluorescent photomicrographs of DA neurons cultured an additional 5 days and labeled for D2 (FIG. 4A, red fluorescence, bar is 50 μm) and for TH (green) and D2 (orange-red) (FIG. 4B, bar is 25 μm).

FIGS. 5A-5E are photomicrographs of DA (4 week) and hNT (5 week) neurons labeled for AHD-2 and TH. FIG. 5A shows clumps of DA (4 week) cells labeled for AHD-2, while FIG. 5B show clumps of hNT (5 week) cells similarly labeled. For FIGS. 5A and 5B bar is 50 μm. FIG. 5C shows DA neurons labeled for ADH-2; FIG. 5D shows DA neurons labeled for TH; and FIG. 5E shows double labeling (arrows) for TH and AHD-2 in DA neurons. For FIGS. 5C-5E the bar is 25 μm.

FIGS. 6A through 6C are photomicrographs illustrating the effects of 4 weeks of RetA and 5 days of LiCl on DA neuronal cells on frequency of TH-expressing cells (6A), TH and PI staining (6B), and bcl-2 expression (6C).

FIG. 7 is a bar graph showing the effects of different doses of lithium [chloride on TH expression in cultured hNT neurons (induced for 5 weeks with RetA).

FIG. 8 is a bar graph showing the effects of different doses of lithium chloride on Bcl-2 expression in cultured DA neurons.

FIG. 9 is a table that shows the effect of lithium chloride on soma size of hNT neurons cultured for 5 days.

FIG. 10 is a table that shows the effect of lithium chloride on neurite growth of hNT neurons cultured for 5 days. In FIGS. 9 and 10, the * denotes significant difference (p<0.01).

FIGS. 11A-11E are photomicrographs of representative control and lithium-treated hNT neurons cultured for 5 days and immunostained for TH. FIG. 11A shows a control culture of hNT neurons and reveals few TH-positive cells. FIGS. 11B and 11C show hNT cells cultured with 1.0 mM (10B) and 3.0 mM (11C) lithium chloride. FIGS. 11D and 11E show the representative morphology of TH-positive hNT cells treated with 1.0 mM (11D) and 3.0 mM (11E) of lithium chloride.

FIGS. 12A-12E are photomicrographs of the distribution, morphological appearance and phenotype of hNT neurons after 5 days in control culture and lithium-treated cultures. FIG. 12A is a phase-contrasts low-magnification photomicrograph showing the distribution of hNT neurons in control/untreated cultures. FIG. 12B is a higher magnification photomicrograph demonstrating that virtually all cultured hNT cells are immunoreactive for GAP43. FIGS. 12C and 12D are low-magnification, phase-contrast photomicrographs of hNT neurons treated with 1.0 mM (12C) and 3.0 mM (12D) concentration of lithium chloride. FIG. 12E illustrates the morphological appearance of GAP-43-labeled hNT neurons treated with 3.0 mM lithium chloride.

FIG. 13 is a bar graph comparing the effects of hNT cells, DA neurons, and LiCl-treated neurons on the lesioned animals' performance of rotations in the PD rat model.

FIG. 14 is a bar graph comparing the mean numbers of surviving hNT, DA, and LiCl-treated neurons at the two implant locations (striatum and substantia nigra).

FIGS. 15A-15D are photomicrographs of FDA-PI stained cells. FIGS. 15A and 20B show NT2 cells at 1 and 5 DIV. FIGS. 15C and 15D show DA cells and 1 and 5 DIV.

FIGS. 16A-16F are photomicrographs of tdt-labeled cells (apoptotic cells). FIG. 16A shows apoptotic nuclei in cultured NT2 cells. FIG. 16B shows a group of DA neurons with apoptotic nuclei. FIG. 16C shows a clump of DA neurons with single or multiple lobes of condensed chromatin. FIG. 16D shows MI apoptotic cells. FIG. 16E shows some dying MI neurons. FIG. 16F shows the positive control (treated with nuclease to generate DNA breaks in cells and staining in all cells).

FIGS. 17A-17F are photomicrographs of DA neuron grafts in the striatum of hemiparkinsonian rats. The DA neurons were immunolabeled with an antibody to Nuclear Matrix Antigen (NuMA). FIG. 17A shows DA neurons pre-cultured for 24 h; FIG. 17B DA+Li chow; FIG. 17C Li+DA; FIG. 17D Li+DA+Li chow; FIG. 17E DA at Thaw; FIG. 17F Li at Thaw. Scale bar is 200 μm.

FIG. 18 summarizes survival of DA neurons after 1 week in vivo. There were significantly more surviving cells in the DA neuron and DA+Li chow groups compared to the Li+DA and the Li+DA+Li chow groups. There were significantly more surviving neurons in the Li at Thaw group than in the other groups. (* p=0.02; ** p=0.05; # p=0.005).

FIGS. 19A-19E show TH-labeled DA neurons in vivo. FIG. 19A shows NuMA-labeled DA Neurons in the striatum (green); FIG. 19B TH-positive cells in the striatum; FIG. 19C TH-positive NuMA-labeled DA Neurons. The asterisks in A-C indicate the same cells within the graft. For FIGS. 19D and 19E, a striking observation with the double immunofluorescent labeling of TH-positive DA neurons was the presence of rarely-seen TH-positive fibers extending from the cell bodies (white arrowheads) within the graft. These were observed in all groups. The scale bar is 10 μm.

FIG. 20 indicates the percentage of grafted DA that expressed TH. There were significant differences in the percentage of the surviving cells that expressed TH, with more double-labeled DA hNT neurons present in the Li at Thaw group. (*p=0.01; ** p=0.05).

FIGS. 21A-21H comprise a photographic montage, demonstrating fiber outgrowth from the DA grafts. Immunohistochemistry identifying human Neuron Specific Enolase (NSE) demonstrated that the DA neurons were developing extensive neuritic processes that extended well into the host striatum after one week in situ. FIG. 21A shows NSE staining of a graft in the striatum. Notice that the most dorsal part of the graft transects the subcortical white matter. The most extensive neuritic outgrowth from the graft occurred in this region. Fibers from these cells were found to extend up to 2 mm from the cell body. FIG. 21B was produced by converting the montage in FIG. 21A to a photographic negative image using Photoshop software (Adobe Systems, San Jose, Calif.) in order to more clearly represent the full extent of fiber outgrowth from the graft. The fibers coursing toward the corpus callosum and in lateral subcortical white matter as well as fibers leaving the ventral portion of the graft to enter host striatum are more clearly visible (arrows). In addition, the patch matrix architecture of the striatum is more apparent in this image. FIG. 21C shows that within the graft the labeled fibers were so dense, that cell bodies were only visible along the margins of the graft. FIGS. 21D-21F shows that within the striatum, processes that exited the graft were mainly found traveling through the fascicles. FIG. 21G is a higher magnification view of fibers in the subcortical white matter shown in FIGS. 21A and 21B (asterisk). FIG. 21H shows that in some cases, fibers were observed crossing the midline of the corpus callosum. Scale bars in FIGS. 21A and 21B are 200 μm. Scale bars in FIGS. 21C-21H are 50 μm.

### DETAILED DESCRIPTION

Dopaminergic neuronal cells are derived from progenitor cells as follows. The progenitor cells are treated with retinoic acid for a time period sufficient to optimize expression of tyrosine hydroxylase. The optimized neuronal cells are further treated with at least one lithium salt or a combination thereof. The DA neuronal cells are harvested. The resulting neuronal cells are highly purified and have a phenotype optimized to produce dopamine, which is diminished in at least one neurodegenerative disease, such as Parkinson's Disease. Optionally, the neuronal cells also are cultured or administered with Sertoli, bone marrow stem, or fetal stem cells.

The six criteria for transplantable cells summarized above are met by DA and hNT neurons. In addition, DA and hNT neurons surprisingly were able to be optimized for stable TH production. TH is vital because it performs the rate-limiting step in production of dopamine. These optimized DA neuron cells have improved dopaminergic properties arising from manipulating the hNT neuron's natural plasticity. These procedures eliminated the need to transfect the cells with exogenous gene constructs.

DA and hNT neurons have the potential to overcome many of the limitations associated with human fetal tissue transplantation, including poor graft survival (5-10%), high tissue variability, and low degree of host re-innervation. hNT neurons have demonstrated excellent graft survival and behavioral improvements in animal models of CNS disorders and also improvement in humans having stable stroke symptoms. There are preliminary data suggesting that hNT neurons may have immunosuppressive properties and produce neuroprotective, neurotrophic factors. Thus, long-term, systemic immunosuppression may not be necessary in humans.

Furthermore, hNT neurons are human cells derived from the human teratocarcinoma NT2/D1 cell line through induction with RetA treatment (Andrews, P W, Damjanov J, Simon D, Banting G, Carlin C, Dracopoli N C, Fogh J. Lab Invest 50:147-162, 1984). During the 6-week retinoic acid induction period, NT2/D1 cells, which share many characteristics of neuroepithelial precursor cells, undergo significant changes resulting in the loss of neuroepithelial markers and the appearance of neuronal markers (Pleasure S J, Page C, Lee V M. J Neurosci 12:1802-1815, 1992; Lee V M, McGrogan M, Lernhardt W, Huvar A. Strategies in Molecular Biol 7:28-31,1994). Several enrichment steps result in the production of >99% pure populations of hNT neurons that are terminally differentiated (Andrews et al, ibid.). They display process outgrowth and establish functional synapses. Thus, mature hNT neurons do not divide, they maintain a neuronal phenotype, and they appear to be virtually indistinguishable from terminally differentiated post-mitotic, embryonic neurons (Pleasure S J, LEE VM J Neurosci Res 35:585-602, 1993).

Definitions:

A dopaminergic deficiency is a condition in which there is a shortage of dopamine. The dopaminergic deficiency may have a variety of causes, including, but not limited to, under-production by dopaminergic neurons, deficit of dopaminergic neurons, or insensitivity of dopaminergic neurons to dopamine. Examples of such conditions include, but are not limited to, Parkinson's disease, schizophrenia, progressive supranuclear palsy (Steele-Richardson-Olszewski Syndrome), and a Dopa-responsive form of torsion dystonia.

“Beneficial effect” is an observable improvement over the baseline clinically observable signs and symptoms. For example, a beneficial effect can include improvements in graft survival, improvements in one or more of the signs and symptoms associated with a dopaminergic deficiency, such as movement or mood.

“Mammal” includes humans and other mammals that would reasonably benefit from treatment, including pets such as dogs and cats.

“Cellular composition” is defined as a mixture of non-fetal live cells, not all of one type, which also contains a balanced electrolyte solution.

“Neuronal cell” is defined as cells giving rise to cell types found in the central and peripheral nervous system. Some of the types of cells include but are not limited to those listed below.

“NT2/D1 precursor cells” as used herein refers to a special cell line available from Layton Bioscience, Inc. (Sunnyvale, Calif.). This cell line has been developed from a previously described human teratocarcinoma cell line (termed Ntera2/clone D1 or NT2 cells) (Andrews et al. Lab. Invest. 50:147-162, 1981). These cells are precursors for “LBS-Neurons” human neuronal cells. NT2/D1 cells are unique among other teratocarcinoma cell lines because these cells act like progenitor cells whose progeny are restricted to the neuronal lineage (Andrews, ibid.) “hNT human neuronal cells” as used herein refers to the special neuronal cell line disclosed in U.S. Pat. No. 5,175,103 to Lee et al. Briefly, NT2/D1 precursor cells are induced to differentiate into neurons by administration of 10 μM RetA which is replenished twice weekly for 6 weeks, after which the cells are replated with special manipulations to become more than 99% pure hNT neurons. These are the cells that are used in the subsequent experiments. Alternately, for human use, there is a cell line manufactured without antibiotics (used in the research grade hNT neurons) and under good manufacturing practices (GMP), which is termed “LBS-NEURONS” human neuronal cells (Layton Bioscience, Inc.).

“Dopaminergic neurons” have a dopaminergic phenotype, including expressing such markers as TH, AHD2, DARPP-32 and D2 dopamine receptor. Dopaminergic neurons are obtained by retinoic acid induction of NT2/D1 cells for about three weeks to about 4 weeks. If the NT2 cells are induced with retinoic acid for four weeks and then replated with mitotic inhibitors, the resulting neurons are herein called DA neurons.

Other cells, including stem cells, are considered to be useful in this invention. The HCN-1 cell line is derived from parental cell lines from the cortical tissue of a patient with unilateral megalencephaly (Ronnett G V et al. Science 248:603-5, 1990). HCN-1A have been induced to differentiate to a neuronal morphology and stain positively for neurofilament, neuron-specific enolase (NSE), which are selective neuronal markers and are negative for glial markers, such as glial fibrillary acidic protein (GFAP) and myelin basic protein. The cells also stain positively for the neurotransmitters gamma-amino butyric acid and glutamate. Subsequently, Poltorak et al. (Cell Transplant 1(1):3-15, 1992) observed that HCN-1 cells survived in rat brain parenchyma and proposed that these cells may be suitable for intracerebral transplantation in humans. Ronnet et al. (Neurosci 63(4):1081-99, 1994) reported that HCN-1 cells grew processes resembling neurons when exposed to nerve growth factor, dibutyryl cyclic AMP and isobutylmethylxanthine.

Neuronal cells also can be administered with macrophages, which have been activated by exposure to peripheral nerve cells. Such activated macrophages have been shown to clean up the site of CNS trauma, for example, a severed optic nerve, after which new nerve extensions started to grow across the lesion. Implanting macrophages exposed to CNS tissue (which secretes a chemical to inhibit macrophages) or nothing at all resulted in little or no regeneration (Lazarov-Spiegler et al. FASEB J. 10:1296-302, 1996).

Xenotransplantation, the use of cells from different species, also is a viable approach to circumventing the limitations associated with human fetal neural transplantation (Galpern W R et al., Exp Neurol 140:1-13, 1996). A phase I clinical trial sponsored by Diacrin, Inc., evaluated transplants of porcine cells harvested from the midbrains of pig fetuses. Fetal pig cells have been implanted into patients with neurodegenerative diseases, such as Parkinson's disease and Huntington's chorea, and intractable seizures, in whom surgical removal of the excited area would otherwise have been performed. Another technique, developed by Cytotherapeutics, Inc., uses encapsulated xenografts of rat PC12 cells. A semipermeable polymer membrane allows diffusion of the small therapeutic molecules (e.g., neurotransmitters) but prevents diffusion of the larger immunogenic molecules. Such cells, if properly screened for retroviruses, could also be used in the inventive method.

Neural crest cells are isolated and cultured according to Stemple and Anderson (U.S. Pat. No. 5,654,183), which is incorporated herein by reference, with the modification that basic fibroblast growth factor (bFGF) is added to the medium at concentrations ranging from 5 to 100 ng/ml in 5 ng/ml increments. Neural crest cells so cultured are stimulated by the presence of FGF in increasing concentrations about 1 or 5 ng/ml. Such cells differentiate into nerve cells, which can be used in the instant invention.

Stem cells of different types can be combined with DA neurons to provide more of the complex architecture that is necessary. There are many examples of stem cells, only a few of which will be mentioned here. Also bone marrow stromal cells, isolated from other cells by their tendency to adhere to tissue culture plastic, have many of the characteristics of stem cells for tissues that can roughly be defined as mesenchymal, because they can be differentiated in culture into neurons (Sanchez-Ramos et al. WO99/56759, Bone Marrow Cells as a Source of Neurons for Brain and Spinal Cord Repair, Nov. 11, 1999).

U.S. Pat. No. 5,753,506 issued May 19, 1998, reveals an in vitro procedure by which a homogenous population of multipotential precursor cells from mammalian embryonic neuroepithelium (CNS stem cells) was expanded up to 10⁹ fold in culture while maintaining their multipotential capacity to differentiate into neurons, oligodendrocytes, and astrocytes. Chemical conditions are disclosed for expanding a large number of neurons from the stem cells. In addition, four factors—PDGF, CNTF, LIF and T3—have been identified which individually generate significantly higher proportions of neurons, astrocytes, or oligodendrocytes. These procedures are intended to permit a large-scale preparation of the mammalian CNS stem cells, neurons, astrocytes and oligodendrocytes. Other sources of stem cells are primates (see U.S. Pat. No. 5,843,780 issued Dec. 1, 1998).

“Inducing agent” includes, but is not limited to, compounds that have the effect of causing NT2/D1 precursor cells to differentiate into hNT neurons, one example of which is retinoic acid. Thus, an inducing agent includes not only retinoic acid in any of it isomers and trans/cis forms, but also similarly active compounds. Other types of inducing agents are mentioned above with different cell types.

“Immunosuppressant” as used herein is a substance which prevents or attenuates immunologic phenomena. For example, such immunologic phenomena include inflammation, autoimmunity, GVHD and graft rejection. Examples of current immunosuppressants include but are not limited to cyclosporine A, cyclophosphamide, prednisone and tacrolimus (Prograf, Fujisawa Inc., Deerfield, Ill.). Optionally, an immunosuppressant can be administered at the time of the transplant. One regimen calls for administering the immunosuppressant for two days, before and on the day of transplantation.

“Vehicle” or “diluent” is a biologically compatible electrolyte solution, such as phosphate buffered saline (PBS) and the like, which is used to suspend the neuronal cells. Optionally, magnesium and calcium salts can be added to the vehicle. One possible diluent is pH-balanced and includes the magnesium, potassium, sodium, chloride, phosphate, acetate and gluconate. Optionally, one may use a commercial balanced electrolyte solution for injection, such as Isolyte S (B Braun McGaw Pharmaceuticals), Ringer's solution, etc. A lithium salt is optionally incorporated therein. This definition also includes any gel or matrix which firms at body temperature and is biodegradable.

As used herein, the term “sample” is meant to refer to one or more treated cells. In preferred embodiments, a sample contains a plurality of cells. According to the present invention, a sample of treated cells is implanted into either a non-human mammal or a human.

By “lithium” is meant generally a lithium salt, wherein the anion includes, but is not limited to, chloride, bromide, carbonate, citrate, or other biologically compatible monovalent anion. In particular, lithium chloride (LiCl) has been used in many of the examples disclosed below. Lithium concentration can be at least about 0.2 mM, about 0.5 mM, about 0.75 mM, about 1 mM, or 1.5 mM. The upper limits of lithium concentration are 5 mM, 3 mM and 2 mM.

“Therapeutic agent” as used herein means the transplanted cells themselves or chemical entities secreted by these cells. Examples of chemical entities secreted by the cells include, but are not limited to dopamine, other neurotransmitters, neurotrophic factors, proteins and hormones. The term transplanted cells is not limited to dopaminergic cells but can include other cells, preferably neuronal cells.

The production of hNT Neurons is an 8-10 week process. All cell culture work has been performed in T-flasks but can be performed in other containers. hNT neurons are induced from NT2/D1 cells following exposure to growth media (e.g., DMEM/F-12 solution, Earle's balanced solution, Hank's phosphate buffer, phosphate-buffered saline) containing 10 μM RetA for about 5-6 weeks. Cells are harvested using trypsin and replated at reduced density. Replate I cultures are maintained in growth media for 2 days and then separated from the accessory cells by gentle selective harvest to give an enriched neuron population. Replate 2 cultures consist of the enriched neuron population from Replate 1, placed in growth medium and replated on Matrigel and treated with mitotic inhibitors. Extended Replate I cultures are plated in media and at 24 hours treated with mitotic inhibitors for 5-10 days. Purified neurons are then selectively harvested using trypsin and formulated for cryopreservation. For some experiments (see examples), the cells were maintained in neuron-conditioned media and allowed to mature in culture. After Ret A induction, hNT neurons constitute approximately 10-20% of the cell population; the remainder are non-neuronal accessory cells. hNT neurons are post-mitotic and no longer capable of dividing; whereas, the dividing accessory cells are mitotically inhibited by the addition of cytosine arabinoside (Ara-C) and fluorodeoxyuridine (FUdR) to culture medium. Harvest results in a purified bulk product of >95% neurons, which is formulated in freezing media and cryopreserved.

### EXAMPLES

### Example 1

### Enhancing Dopaminergic Properties of hNT Neurons

A series of studies evaluated the dopaminergic potential of other neuronal precursor cell lines, the optimal time for RetA induction of TH during the differentiation of the NT-Neurons, and the process for stabilization of TH during replate purification.

### Example 1.A

### The Effect of Maturation in Replate of hNT-Neurons on TH Expression

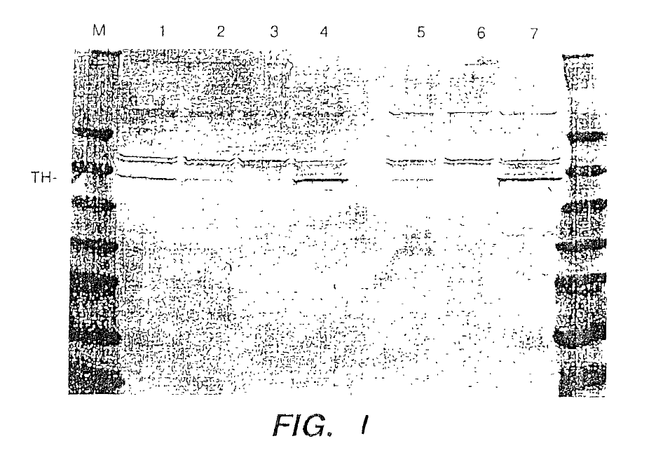

A study examined the effects of in vitro maturation and extended replate of hNT neurons on TH levels. After the hNT Neurons have developed during the 5 to 6-wk RetA induction, cultures are routinely replated and treated with mitotic inhibitors in the process of purifying the neurons (see above). The hNT Neurons were maintained in culture and allowed to mature either as a replate-I culture or as an enriched replate-II culture. To optimize TH expression, neurons were purified after culturing under different replate conditions, and the levels of TH compared in the Western Blot assay. Samples were prepared from purified hNT Neurons that had been treated with inhibitors for 7 days as replate-I or replate-II cultures, and then maintained in growth media for a total of 1, 2, or 3 weeks of maturation. Extracts of the purified neurons were analyzed by Western Blot (FIG. 1). The level of TH expression decreased dramatically with maturation in culture, and it was no longer detectable after 2 weeks in replate-II or after 3 weeks in replate-I. The levels of TH found in the replate-I neurons not only were significantly higher, but also were expressed for a longer period. Control Western Blots (not shown) were developed for each assay using an anti-Tau monoclonal to confirm that 1) equivalent numbers of hNT neurons were loaded, and 2) the samples were not degraded.

### Example 1.B

### Optimization of RetA Induction for TH Expression in Neurons

TH expression paralleled the early development of neurons and was evident by 3 to 4 weeks of RetA induction. Since the preliminary results showed that the TH expression level had been greatly reduced upon maturation of hNT neurons for 2 to 3 weeks after replate, a strategy was designed to determine if more TH was produced by less matured neurons. Possibly the hNT neurons that were produced after 5-6 weeks of RetA induction, which was optimal for the yield of cholinergic neurons, have been committed to down-regulate TH. To optimize production of neurons for expression of TH and dopaminergic properties, a time course of RetA induction was performed; and the TH levels in purified replate-I neurons from different RetA inductions were analyzed.

The NT2 precursor cells were induced with RetA for 4 weeks (DA neurons) or 5 or 6 weeks (hNT neurons). The cultures were replated and after 24 hours mitotic inhibitors were added and maintained for 7 days. The cells were refed with growth media, and the neurons harvested either after 1 day or after an additional 7 days. The extracts were prepared for denaturing SDS-PAGE, and samples containing the equivalent of 10⁶ cells/lane were transferred to Western blots.

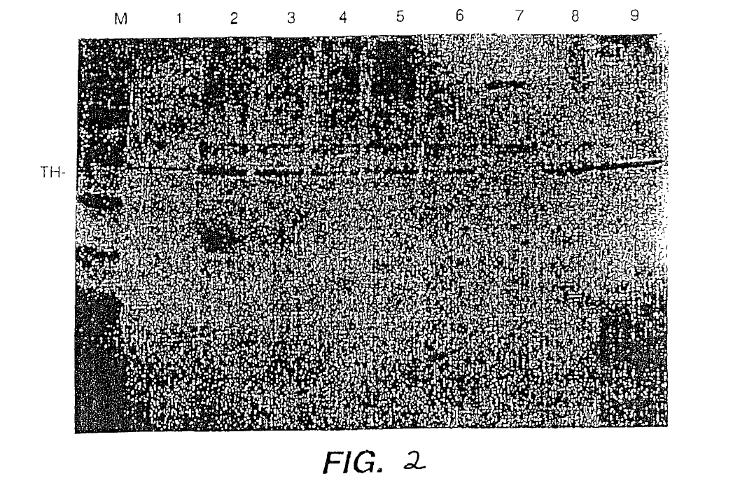

The dramatic effect of RetA induction times on TH expression levels in the neurons is shown in FIG. 2. The expression of TH was found to be the highest in the DA neurons that were purified from the 4-week RetA Induction (Lane 2). The TH levels decreased significantly in purified hNT neurons RetA induced for 5 and 6 weeks (compare Lane 2 to Lanes 3 and 4). The loss of TH expression becomes even more evident in the 2-week matured DA neuron (4-wk RetA) samples (Lane 5) compared to hNT neurons (5- and 6-week RetA) (Lanes 6 and 7). These results demonstrate that there is an optimal RetA induction period of greater than 3 but less than 5 weeks, perhaps peaking at 4 weeks. These results also confirm that subsequent maturation in vitro reduces TH expression, even in the high-expressing immature DA neurons (4-week RetA).

### Example 2

### Comparison of DA and hNT Cryopreserved Neurons After Thawing and Plating in Culture for 5 Days

To further characterize dopaminergic neurons, NT2/D1 cells were treated with retinoic acid for 4 weeks (DA neurons) or 5 weeks (hNT neurons), were cryopreserved, stored frozen, thawed, and then cultured for 5 days. Additional testing was performed to determine if the DA neurons had other biochemical attributes of the substantia nigra (SN) dopaminergic (DA) neurons, including tyrosine hydroxylase (as discussed above), dopamine membrane transporter for reuptake of dopamine from the synaptic cleft, D2 dopamine receptor (D2) for regulating dopamine release and aldehyde dehydrogenase (AHD2). AHD2 has been found in a subpopulation of dopaminergic neurons of the mesostriatal and mesolimbic system shortly after the appearance of TH but not in other dopaminergic neurons.

DA neurons and hNT neurons (Layton Bioscience, Inc., Sunnyvale, Calif.) had been stored at −180° C. prior to use. The cells were thawed, resuspended in the medium containing DMEM (Gibco) and 10% fetal bovine serum (Gibco), and were plated on poly-L-lysine coated 8-well chamber slides at a concentration of 100,000 cells/cm². After 24 hr the plating media was switched to DMEM: F12 containing 0.1% ITS (Sigma) and gentamicin (50 μg/ml, Sigma). Cultures were maintained for an additional 4 days, then rinsed with 0.1 M PBS and fixed with 4% paraformaldehyde. However, some DA neurons were fixed after the initial 24 hr culturing to assess the effect of thawing on TH expression.

For immunochemistry, cultures were thoroughly washed in 0.1M PBS and incubated in 10% serum from the host secondary antibody with 0.03% Triton X-100 in PBS for 1 hr. Cultures were then incubated for 24 hr in the same solution containing one of the following: 1) primary antibody against TH (1:4000, mouse monoclonal antibody, Diasorin, Stillwater, Minn., or 1:500, rabbit polyclonal antibody, Pel-Freez™, Rogers, Ariz.), b) DAT (1:5,000, rat monoclonal or 1:500, rabbit polyclonal antibody, Chemicon, Temecula, Calif.), c) AHD-2 (1:5000, rabbit polyclonal antibody, courtesy of Dr. Ron Lindahl, University of SD), d) D2 receptor polyclonal antibody (1:1000, rabbit polyclonal antibody, Chemicon) or a mixture of primary antibodies, including antibodies to TH combined with an antibody to DAT, D2 or AHD-2.

For single staining, the slides were washed with PBS and incubated for 1 hr in the appropriate biotinylated secondary antibody (1:200, Vector, Burlingame, Calif.). The antibody complex was developed using avidin-biotin kit (ABC-Elite kit, Vector), and the final product was visualized either by using 3,3′-diaminobenzidine (DAB; ImmunoPure Metal Enhanced DAB, substrate kit; Pierce, Rockford, Ill.) or VIP (Vector, peroxidase substrate kit, Burlingame, Calif.). For immunofluorescent staining, TH was visualized using fluorescein isothiocyanate (FITC) conjugated to goat anti-mouse IgG (1:500, Alexa™, Molecular Probes, Inc., Eugene, Oreg.) or rhodamine conjugated to goat anti-rabbit IgG (1:200, Jackson ImmunoResearch), AHD-2 and DAT were visualized using rhodamine conjugated to goat anti-rabbit IgG (1:200). Finally slides were rinsed in 0.1 M PBS and cover-slipped using 95% glycerol or Vectashield (Vector). For control sections, one or both of the primary antibodies were omitted. The enzyme-linked immunostained slides were examined using an Olympus BH-2, while immunofluorescence-stained slides were analyzed and photographed using the Olympus BX 40 and BX 60.

Prior to cell counts, 8-well slides were stained with the appropriate primary antibody and visualized with DAB. Through 20× objective and a photographic frame (field=0.3 mm²), 16 predetermined sites per well (4-8 wells/plating/marker) were viewed to count the number of labeled and unlabeled cells. Percentages were determined as a ratio of labeled cells/total cells multiplied by 100. The mean values ±SEM for every marker were determined from three independent cultures. The differences between the dopaminergic markers between DA and hNT neurons were compared using Student's t-test.

The co-localization of TH with other dopaminergic markers was evaluated from fluorescently labeled slides viewed through single or double fluorescent filters. The co-expression of two markers was determined in randomly selected fields (n=30-40/plating), under 40× magnification. Cells that were positive for DAT, D2, or AHD-2 alone or positive for both markers (TH/DAT, TH/D2, or TH/AHD-2) were recorded.

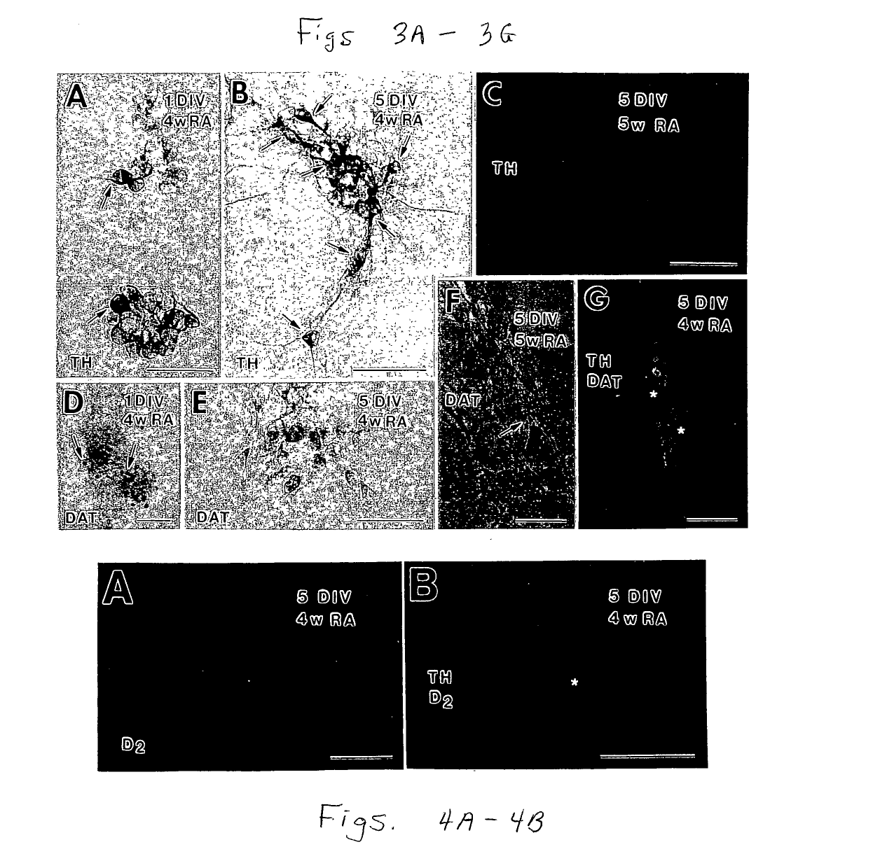

24-hr plated cells had many clumps of small round cells evenly spread over the culture dish. Numerous cell bodies stained for TH; but there were only a few extended, short processes that were not very prominent (FIG. 3A). Representative cultures contained 44.3-64.9% TH+ neurons. DAT was also found and had a more variable staining because of diffuse label in some cells and a more common punctate/granular appearance in cell somas and processes (FIG. 3D).

After 5 days in vitro (DIV), cell morphology substantially changed. Individual cells and clusters of varied sizes revealed TH+ positive cell bodies and long, branching processes spread toward neighboring clumps (FIG. 3B). Quantitatively among 5 DIV cultures, the percentage of TH+ cells varied between 33.3% and 87.2% which was not significantly different from TH+ cells in 1 DIV cells. However, TH expression did differ significantly (p<0.01) between DA neuron and hNT neuron cultures (58.7% and 14.8%, respectively) (FIGS. 3B and 3C). Interestingly, the percentages of DAT+, D2+, and AHD-2+were equally high for DA and hNT neurons (79.9% to 91.2%). FIGS. 3E and 3F show DAT staining of DA and hNT cells, respectively. See Table 1.

TABLE 1
Dopaminergic Phenotype of DA and hNT neurons (5 DIV)

| RA treatment | TH | DAT | D2 | AHD-2 |
|---|---|---|---|---|
| DA neurons | 58.7 ± 3.7 (5419) | 79.9 ± 2.2 | 91.2 ± 3.9 | 82.7 ± 9.2 |
| | | (3042) | (1192) | (2102) |
| hNT neurons | 14.8 ± 2.3 (2176)* | 79.2 ± 2.9 | 89.9 ± 0.8 | 81.9 ± 1.9 |
| | | (2631) | (1229) | (3140) |

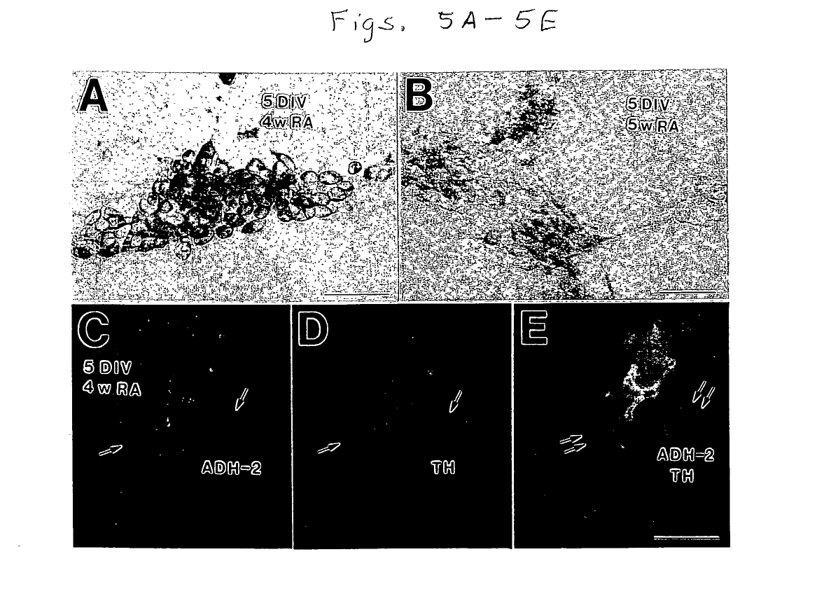

Nearly all TH+ neurons (93%) also were DAT+. Moreover, 53% of all D2+ neurons also stained for TH, indicating TH+cells also have D2 dopamine receptors to regulate dopamine release. D2 staining alone of DA neurons is shown in FIG. 4A; combined TH and D2 staining of DA neurons is shown in FIG. 4B. Virtually all TH+ cells were also AHD-2 positive, indicating that DA neurons had a phenotype typical of the cells involved in Parkinson's disease (a subpopulation of dopaminergic neurons of the mesostriatal and mesolimbic system). FIGS. 5A-5E show AHD-2 staining of DA and hNT cells with and without TH staining. FIG. 5A shows 5 DIV DA neurons, more of which stained for ADH-2 (chromogen DAB) than did the hNT neurons in FIG. 5B (chromogen VIP). For FIGS. 5A and 5B the bar is 50 μm. FIG. 5C shows ADH-2+ DA neurons (red fluorescence, arrows) visualized by a rhodamine-conjugated secondary antibody. FIG. 5D shows TH+ DA neurons visualized by a secondary antibody conjugated to fluorescein. FIG. 5E shows double-labeled (TH+/AHD-2+) DA neurons. For FIGS. 5C-5E the bar is 25 μm.

In conclusion, these cells have all the necessary cellular machinery to produce functional dopaminergic neurons and therefore are a better choice as an alternative tissue source to fetal ventral mesencephalon or other dopamine-producing cells. TH alone is not a sufficient marker for a dopaminergic cells since TH also participates in synthesizing other catecholamine neurotransmitters (epinephrine and norepinephrine). Dopamine neurotransmission also requires presynaptic release of dopamine and its reuptake through the sodium-dependent DAT. Thus, the DAT activity determines the synaptic concentration of dopamine and the level of dopamine receptor stimulation.

### Example 3

### Comparison of Fresh and Thawed DA Neurons

“Fresh Cells” had undergone Replate II treatment (see above) and were never frozen. Prior to freezing, other cells underwent Replate I treatment. Control and lithium-treated cells (fresh or frozen) were cultured for five days and scored for TH immunoreactivity (see above method). The TH-immunostained slides were counterstained with propidium iodide to identify dead cells. Total numbers of TH+ and propidium iodide-positive (PI+) neurons were counted in control and lithium-treated cultures from standardized fields at 20× magnification, as described above.

TABLE 2
TH Expression in cultured (fresh Replate II) DA neurons

| LiCl Dose | TH+ cells | Unlabeled Cells | Total Cells |
|---|---|---|---|
| Control | 256 (56.1%) | 209 | 465 |
| 1.0 mM | 318 (85.3%) | 53 | 371 |
| 3.0 mM | 452 (76.8%) | 138 | 590 |

TABLE 3
TH Expression in cultured (thawed) DA neurons

| LiCl Dose | TH+ cells | Unlabeled Cells | Total Cells |
|---|---|---|---|
| Control | 649 (70.4%) | 272 | 921 |
| 1.0 mM | 846 (79.1%) | 224 | 1070 |
| 3.0 mM | 623 (70.4%) | 262 | 885 |

Note that there are much higher numbers of total thawed DA neurons than of total cells processed by Replate II. Western analysis indicates that the levels of TH in fresh Replate II cultures were lower than those found in thawed, frozen neurons (Table 3). Exposure to 1.0 mM LiCl significantly increased the number of TH+ cells to 80-85%. When no primary antibody was added to the negative control cultures (primary delete), the control cultures were immunonegative.

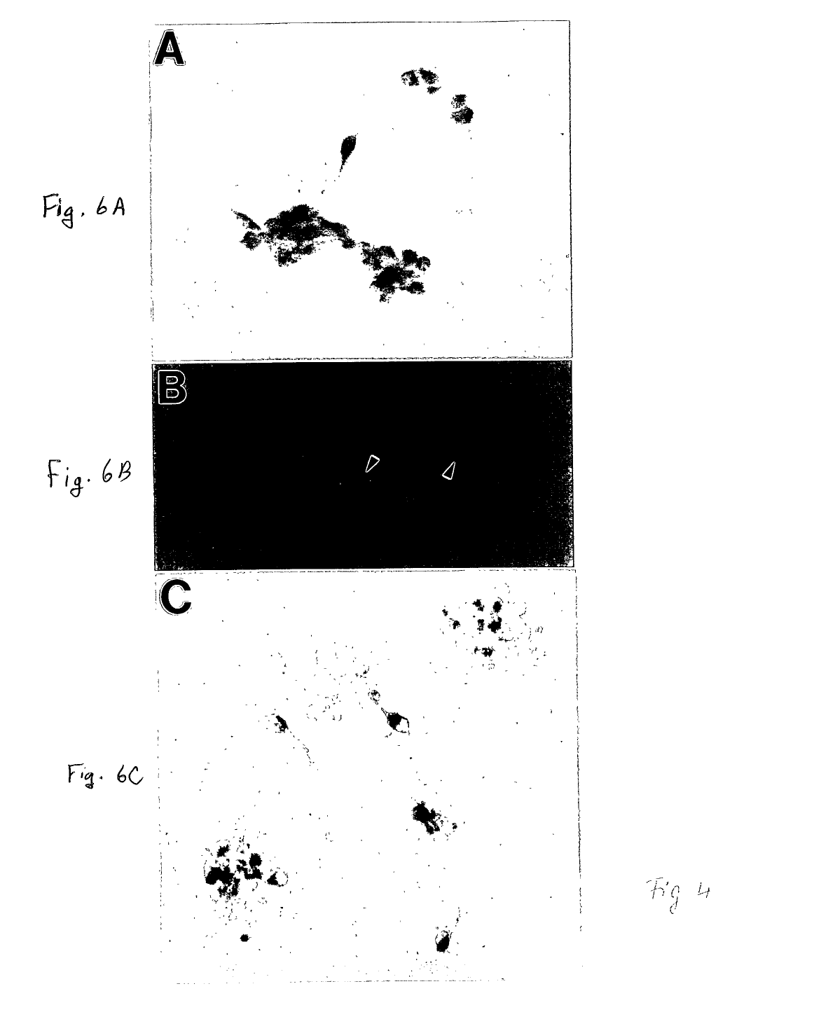

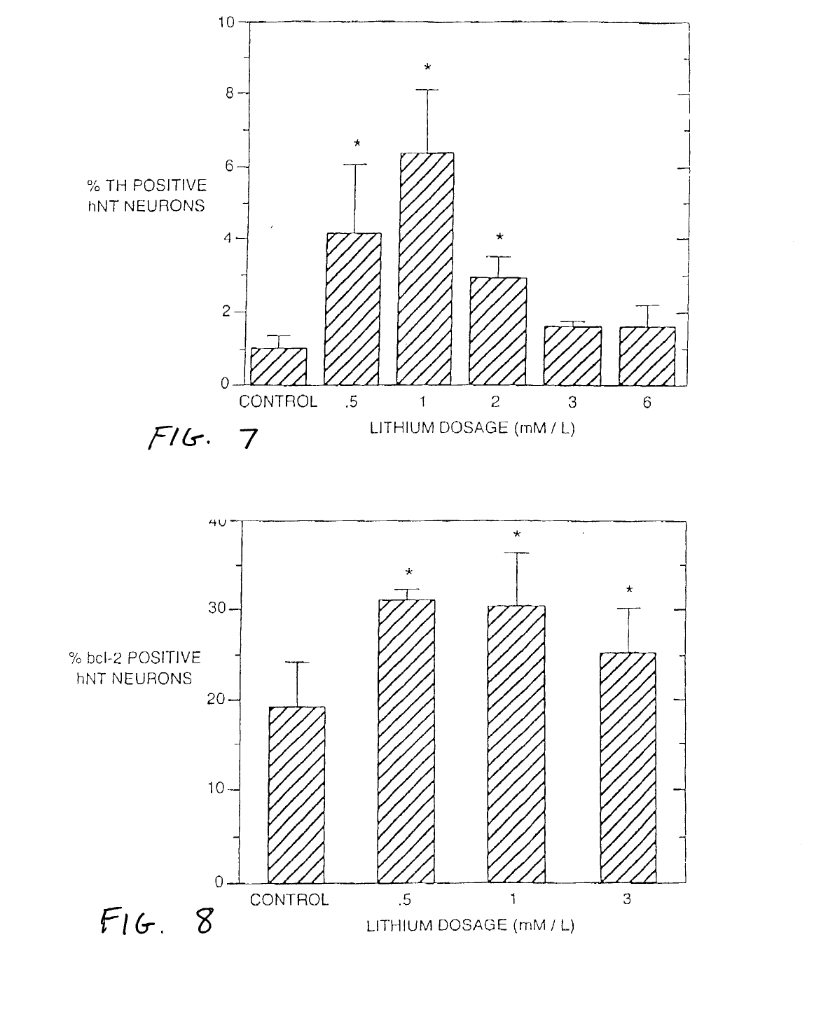

Photomicrographs (FIGS. 6A-6B) show representative 5 DIV DA neurons that had been induced for four weeks with RetA. FIG. 6A is a control culture of DA neurons immunostained with antibodies to TH which had a significantly higher number of TH+ cells in comparison to controls treated for six weeks with RetA (See FIG. 7 for controls). FIG. 6C is a control culture of DA neurons immmunostained with antibodies to bcl-2 to demonstrate the co-localization of an anti-apoptotic gene with TH expression.

### Example 4

### Lithium Induction of bcl-2 Expression

Because the proto-oncogene bcl-2 has been shown to protect a variety of cell types from programmed cell death, it is often considered an inhibitor of apoptosis (Sentman et al. Cell 67(5): 879-88, 1991). Lithium-treated cells were tested for the involvement of bcl-2 that could help protect hNT neurons from apoptosis. First, the immunocytochemical expression of bcl-2 protein in hNT cells cultured for 5 days with 0.5 and 3.0 mM LiCl was evaluated. Immunostaining was performed as described for TH (above), except that the monoclonal antibody to bcl-2 (Ab-1, 1:50, Calbiochem, Oncogene Research Products, Cambridge, Mass.) was used. FIG. 8 summarizes the effects of different doses of LiCl on bcl-2 immunostaining. The number of bcl-2+ cells per well versus total number of cells in experimental (0.5 and 3.0 mM LiCl) and control groups were compared. Among control cells, 19.14% (288/1504) were bcl-2+; among 0.5 mM lithium-treated cells, 31.62% (278/879) were bcl-2+; and among 3.0 MM lithium-treated cells, 29.50% (562/1903) were bcl-2+. These results indicate that lithium enhances bcl-2 expression in hNT cells and thus may act as a neuroprotective agent.

### Example 5

### Effect of Lithium on Size and Neurite Outgrowth of hNT Neurons

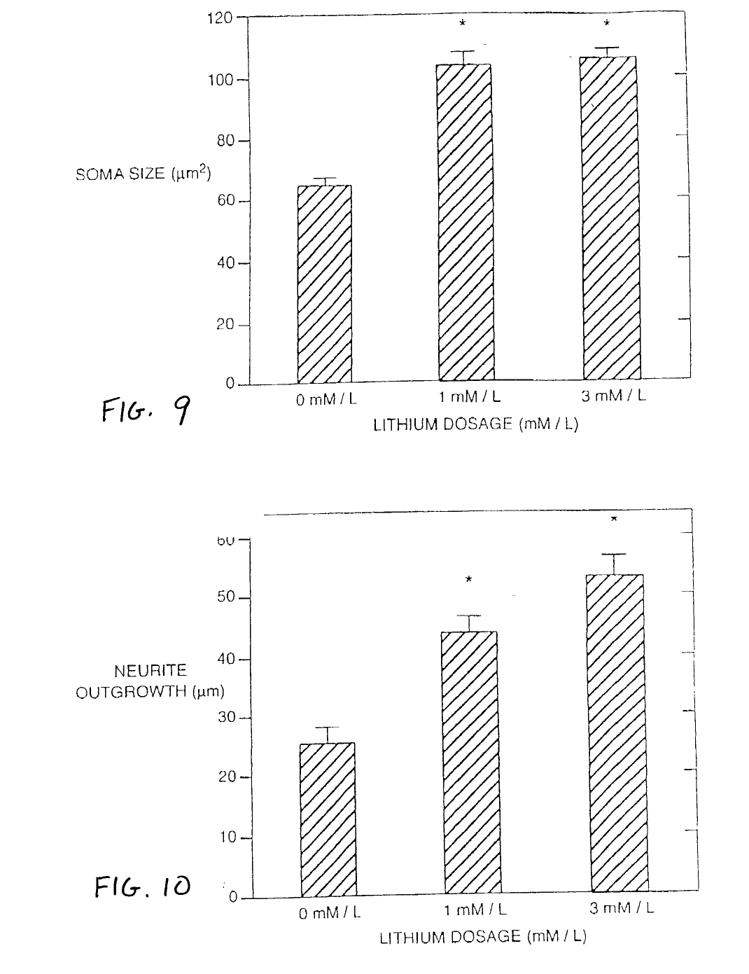

The same series of slides immunostained for TH were employed to measure the soma size (μm²) and neurite outgrowth (μm) of appropriately 50 TH+ cells per representative culture experiment using a computerized image analysis program (Image-Pro Plus, Media Cybernetics, Inc., Silver Springs, Md.) at 20× objective. The results in the morphological assessment study are reported as mean ±SEM and were analyzed using Student's t-test. The size of the TH+ cell bodies increased significantly (p<0.01) after application of 1.0 and 3.0 mM dose of lithium chloride, ranging from 33.8 μm² to 103.3 μm² (mean=64.1±2.5 μm²) in the control group and from 53.09 to 183.3 μm² (mean=103.2±2.7 μm²) and from 61.4 to 165.8 μm² (mean=104.8±3.2 μm²) in 1.0 and 3.0 mM lithium-treated groups, respectively (FIG. 9). Soma sizes in 5DIV NaCl or KCl-treated cultures were not significantly different from control.

The second parameter characterizing the effect of LiCl on the development of TH+ hNT cells was neurite outgrowth. The length of processes in controls has a mean of 25.02±2.9 μm, while in both groups exposed to LiCl significantly (p<0.01) longer processes were found (FIGS. 6A-6B). In cultures treated with 1.0 mM dose of LiCl, the lengths ranged between 12.2 μm and 87.3 μm (mean=43.4±2.8 μm); and in the group treated with 3.0 mM concentration, the lengths varied between 20.3 μm and 128.1 μm (mean=52.9±3.8 μm). Neurite outgrowth in NaCl and KCl-treated 5DIV did not significantly differ from control values. These results clearly demonstrated that morphological development was significantly enhanced in TH+ hNT cells treated with both LiCl concentrations.

Soma size and neurite outgrowth were also measured in hNT cells maintained in culture for 10 days and treated for 5 days with the most effective dose of LiCl (1.0 mM). The mean soma size of TH+ hNT cells was significantly larger (102.8+2.51 μm²) (p<0.01) than in 5 days in cultures, but did not differ significantly from the mean value of the lithium-treated group (103.2±2.7 μm²). The mean length of neurite processes in controls was 24.8±2.4 μm which was not different from younger (5DIV) control cultures but significantly different from 10 day LiCl-treated group (55.5+5.1 μm). In addition, as a result of LiCl treatment, numerous TH+ cells revealed multiple branching processes with varicosities. Collectively, these results suggested that TH-converted hNT cells responded to LiCl treatment by enhancing morphological maturation at both time points studied.

Morphometric analysis revealed that TH+ cells in cultures exposed to lithium resulted in significantly enlarged soma size and longer neurites, as well as a higher degree of neuronal complexity. Taken together, our results suggested that the most effective concentration of lithium (1.0 mM) was adequate to induce TH expression and morphological development of cultured hNT neurons.

### Example 6

### Effect of LiCl on Viability of hNT Neurons

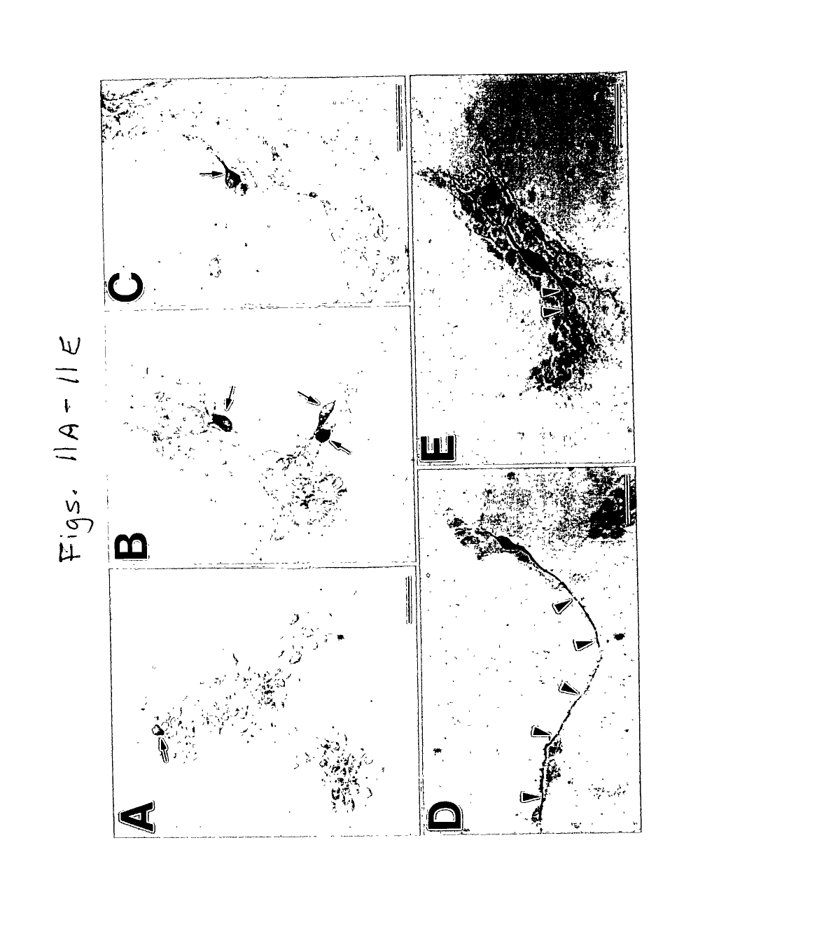

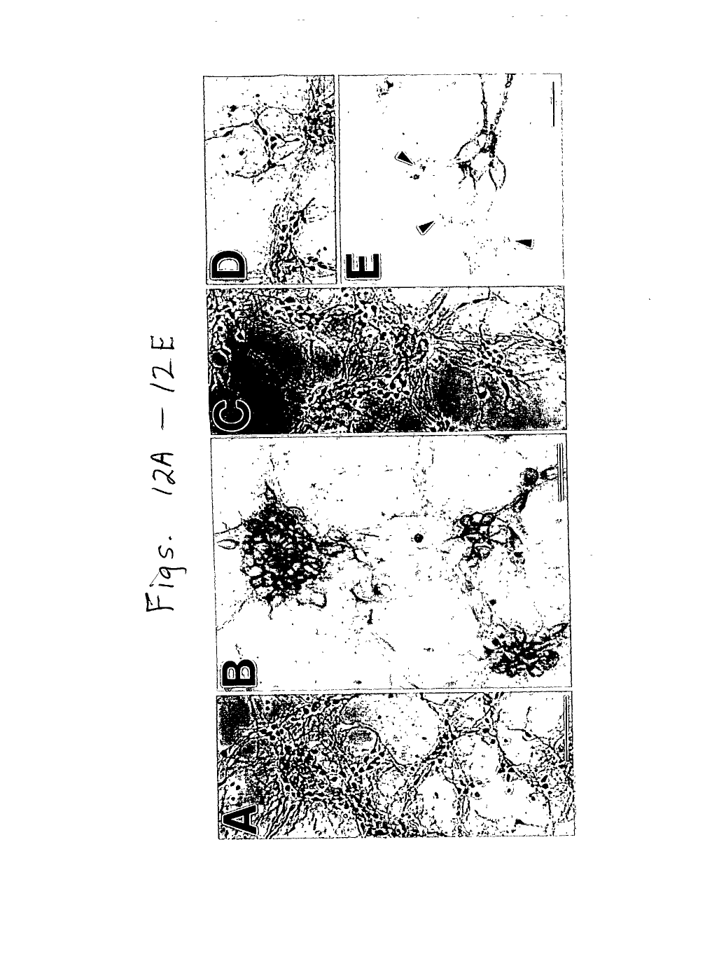

The effect of LiCl on survival of hNT neurons was also evaluated from cultures fixed and immunostained for neuronal marker, growth-associated protein (GAP-43). The actual counts were obtained by placing the photographic frame of the microscope over five randomly chosen fields (field size=0.2 mm²) in each well at 200× magnification. The mean number of GAP-43-positive cells per field was calculated from 4 wells per condition. This type of immunostaining was selected to confirm neuronal phenotype and to facilitate the neuronal counts. Morphologically, GAP-43+ hNT neurons usually exhibited round or oval perikarya and neurites including growth cones (FIGS. 11A-11E). In FIG. 12B, higher magnification shows that virtually all cultured hNT cells are GAP43+, and thus have a neuronal phenotype. The scale is 50 μm.

Independent of survival time or lithium dose used, hNT neurons were aggregated into tightly or loosely packed clusters frequently interconnected with each other (FIGS. 12C, 12D). Low-magnification phase contrast photomicrographs of hNT neurons treated with 1.0 mM (FIG. 12C) and 3.0 mM (FIG. 12D) concentration of LiCl. In both experimental groups the aggregation pattern and morphological appearance of cultured hNT neurons was similar to cultures unexposed to LiCl treatment. The scale is 100 μm in FIG. 12A. FIG. 12E shows the appearances of GAP-43+ hNT neurons treated with 3.0 mM LiCl demonstrating GAP-43+ cell bodies with prominent growth cones (arrowheads) similar to those observed in cultures not supplemented with LiCl. The scale is 25 μm.

In addition, GAP-43 immunostaining facilitated the neuronal counts on these cultures, whose aggregates hampered the cell counting if unstained. Typically, in the 5 DIV hNT control cultures, the number of viable neurons varied between 110-140 per field, which was similar to cultures receiving 1.0 and 3.0 mM LiCl. In control 10 DIV cultures and cultures treated with 1.0 mM LiCl, the individual counts per field ranged from 110-150; and in the group treated with 3.0 mM LiCl counts varied between 100-140/field. These counts were not statistically significantly different from the control values. When the mean number of neurons/field was used to calculate the total number of neurons per well in control cultures, it was shown, that there were about 50,000-54,000 cells/well at both 5 and 10 days. As the initial cell plating in all groups was 89,000 cells/well, this result suggests that there was an approximately 30-40% loss of cells, likely caused by their detachment from the surface of the dish. Taken together, these findings indicated that the presence of 1.0 or 3.0 mM of LiCl had no deleterious effect on the survival of hNT neurons in vitro.

The second important finding of this study was that the most effective TH-inducing dose of LiCl (1.0 mM) was not detrimental to cultured hNT neurons. This dose is within the range of lithium therapeutic concentrations (0.5-1.0 mM) (Johnson, Aust N Z J Psychiatry 21(3):356-65, 1987); and in addition to being employed in treatment of mood disorders, LiCl is a neuroprotective agent against a variety of neurological deficits. A neuroprotective effect of chronic LiCl administration on focal cerebral ischemia was recently shown by Nonaka and Chuang (Neuroreport. 9(9):2081-4,1998). The authors assumed that chronic LiCl-induced neuroprotective benefit is probably due to its ability to attenuate excessive calcium influx mediated by NMDA receptors. They also reported that chronic LiCl treatment (at therapeutically relevant concentrations of this drug, or about 1.3 mM) robustly protected cultured CNS neurons against excitotoxicity mediated by NMDA receptors (Nonaka et al., J Pharmacol Exp Ther 286(1):539-47, 1998). An anti-apoptotic effect of LiCl on cultured cerebellar granule cells has also been reported after application of anticonvulsant (Nonaka et al., 1998).

### Example 7

### Production of LiCl-Induced DA-Neurons

In small-scale cultures, expression of TH in the 4-week DA-Neurons was optimal after the neurons were replated in serum-free media containing 1 mM LiCl for 5 to 7 days. To determine the conditions for production of LiCl-induced DA-Neurons, a series of LiCl treatments were designated to evaluate conditions for optimal TH expression.

The purpose of this experiment was to determine the effect of LiCl treatment during the Replate I mitotic inhibition on DA neurons, which occurs just before harvest of the DA Neurons. Replate cultures were treated with 1 mM LiCl in the presence of mitotic inhibitors (FUdR & AraC) for all 7 days of replate or only during the last three of the 7 days (prior to harvest). The 3-day LiCl treatment was also evaluated without mitotic inhibitors and in serum-free media (+ITS). After treatment the neurons were selectively harvested and processed, and TH levels were analyzed using Western Blots. The 7-day-LiCl DA Neurons contain about 50% higher levels of TH than the 7-day inhibitor-only control. Surprisingly, the 3-day-LiCl neurons also expressed levels of TH comparable to the control, with the uninhibited sample expressing somewhat more TH. The neurons harvested from serum-free media expressed significantly less TH. Serum-free neurons may not have developed as well and may have been contaminated with accessory cells.

DA neurons treated with LiCl expressed comparable levels of TH to those of DA neurons maintained in DMEM/F-12 growth medium with inhibitor. DA neurons with 7 days of LiCl treatment and with 3 days of LiCl treatment had similar levels of TH. The weaker signals for some cells may be due to higher contamination with accessory cells. During harvest of some flasks, the accessory cell layer came off more rapidly than it did from other flasks.

### Example 8

### Comparison of DA and hNT Neurons in PD Rat Model

For 7 days before surgery, 27 female Wistar rats (Charles River, St. Constant, Quebec, Canada), weighing 200-225 g, were housed two per cage with food and water ad libitum and acclimatized for the animal care facility. All animal procedures were in accordance with the guidelines of the Canadian Council on Animal Care and the University Council on Laboratory Animals. Rats were anesthetized intramuscularly with 3.0 ml/kg of a ketamine-xylazine-acepromazine anesthetic mixture (25% ketamine hydrochloride; Ketalean, MTC Pharmaceuticals, Cambridge, Ontario), 6% xylazine (Rompun, Miles Canada, Etobicoke, Ontario); 2.5% acepromazine maleate (Wyeth-Ayerst Canada, Montreal, Quebec); in 0.9% saline. Then rats received two stereotactic injections of 6-OHDA (Sigma Chemical Company, Chicago, Ill.) (3.6 μg of 6-OHDA HBr/μl in 0.2 mg/ml of L-ascorbate in 0.9% saline) into the right ascending mesostriatal dopaminergic pathway at the following coordinates: 1) 2.5 μl at anteroposterior (A/P)=−4.0, mediolateral (M/L)=−1.2, dorsoventral (D/V)=−7.8, toothbar=−2.4; and 2) 3.0 μl of 6-OHDA at A/P=−4.0; M/L=−0.8; D/V=−8.0; toothbar=+3.4. The rate of injection was 1 μl/min with the cannula being left in place for 5 min before being slowly retracted.

Animals were allowed to recover for two weeks in the animal care facility before given an amphetamine challenge (5.0 mg/kg ip), and their rotational scores were collected over a 70 min period using a computerized video activity monitor programmed for rotational behavior (Videomex®, Columbus Instruments, Columbus, Ohio). Only animals exhibiting a mean ipsilateral rotational score of eight or more complete full body turns per minute were included in the implant study.

Sixteen animals received double grafts of hNT neurons, seven received DA neurons, and 4 received LiCl-pre-treated DA-neurons. Three types of neurons were obtained from Layton Bioscience, Inc.: hNT neurons (6 wk RetA); DA neurons (4 wk RetA); and LiCl-pretreated DA-neurons, all of which were stored at −180° C. until the time of transplantation. Two weeks following 6-OHDA lesions, rats were chosen for transplantation if they had a mean rotational score of 8 full body turns per minute. Beginning on the day of transplantation, each animal received 10 mg/kg of cyclosporine A ip for the duration of the experiment. Prior to transplantation, the neurons were quickly thawed by placing the frozen vials in a water bath at 37° C. The neurons were then washed three time in DMEM/0.05% DNase (Sigma Chemical) and centrifuged. The cells were resuspended, and the cell viability and suspension concentration were calculated. The trypan blue dye exclusion method, which stains dead cells blue and fails to stain live cells, was used to assess cell viability.

The cell suspensions were stereotactically injected both intrastriatally and intranigrally using a technique previously described (Mendez and Hong, Brain Res 778: 194-205, 1997; and Mendez et al., 1996, ibid.). A specially designed capillary tip micropipette with an outer opening diameter of 50-70 μm is attached to a 2 μl Hamilton syringe and used to stereotactically implant the desired number of cells at a rate of 100 nl/min into both the SN and striatum (400,000 cells/site). Each animal received a total of about 800,000 cells. Injection of the cells into the dorsolateral striatum occurs at the following coordinates: 1) A/P=+1.3, M/L=−2.1, D/V=−5.5 and −4.3; 2) A/P=+0.6, M/L=−2.9; D/V=5.5 and −4.3; and 3) A/P=+0.3, M/L=−3.7, D/V=−5.5 and −4.3; toothbar=−3.3; coordinates from bregma and dorsal surface of the skull and the SN at the following coordinates: 1)A/P=−4.8, M/L=−2.0 D/V=−8.3 and −8.1; 2)A/P=−5.0, M/L=−2.3, D/V=−8.2 and −8.0; and 3) A/P=−5.3, M/L=−2.6, D.V=−8.1 and −7.9; toothbar=−3.3; coordinates from bregma and the dorsal surface of the skull.

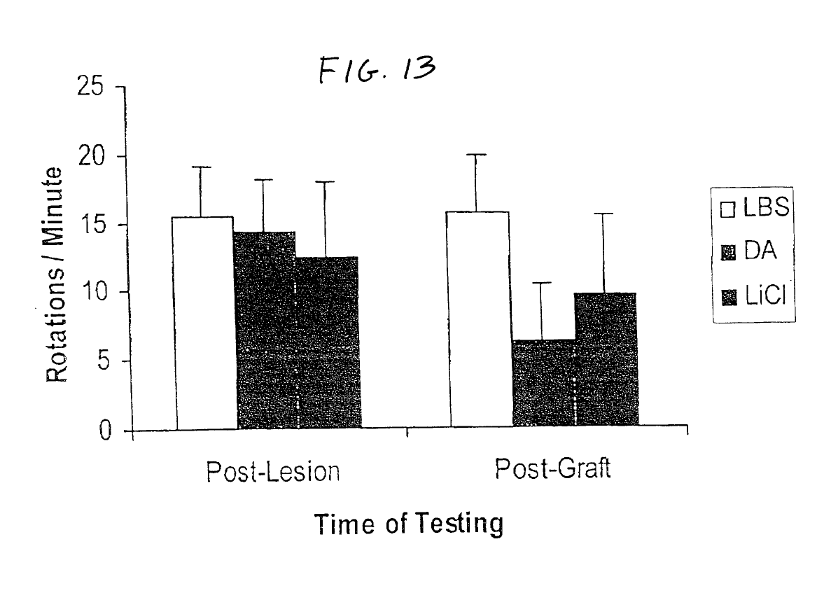

At 3- and 6-wk post-transplantation, the rats were tested for rotational behavior. Comparison data are shown in FIG. 13 for post-lesion and 6 wk post-transplantation. The mean and standard deviation (SD) rotations per minute with amphetamine challenge were recorded as described above. Data for hNT neurons are shown in the white bars, for DA neurons in the gray bars, and LiCl-pre-treated DA neurons in the black bars. There was no change for the hNT neuron-treated rats, as expected. A reduction of rotational behavior was observed in DA-neuron and LiCl pre-treated DA-neuron groups.

At about 6 wk post-transplantation, the rats were euthanized with an overdose of anesthetic (supra) and perfused transcardially with 100 ml of 0.1 M phosphate buffer (PB), followed by 250 ml of 4% paraformaldehyde in 0.1M PB for 10 min. The brains were then removed from the cranium and fixed with 4% paraformaldehyde in 0.1M PB overnight before being stored for 24 hr in PB saline (PBS) containing 30% sucrose. With the freezing microtome, 40 μm coronal sections were made and stored in Millonig's solution (6% sodium azide in 0.1 M PB) until immunohistochemical processing of the sections could be performed. Following processing, sections were mounted in 0.1 M PB on gelatin-coated slides and coverslipped with Permount® mounting medium (Fisher Scientific).

Staining for the presence of tyrosine hydroxylase (TH) was performed using the primary rabbit anti-TH antibody (Ab; 1:2500; Pel-Freeze Biologicals, Rogers, Ariz.) and the ABC-kit (Vector Laboratories Canada, Inc., Burlington, Ontario, Canada). For this procedure the sections were pre-washed for 10 min in a solution of 10% methanol and 3% hydrogen peroxide and blocked in PB containing 0.3% Triton X-100 and 5% NSS for 1 hr. The sections were removed and incubated in a 1:2500 solution of rabbit polyclonal anti-TH Ab for 16 hrs. To visualize Ab binding, 1:500 biotinylated swine anti-rabbit IgG Ab (DAKO Diagnostics Canada, Inc., Mississauga, Ontario, Canada) was used, followed by a streptavidin-biotinylated HRP complex kit. The peroxidase activity was visualized by the addition of DAB. The sections were then washed in 0.1 M PB before mounting.

Staining for the presence of neural cell adhesion molecule (N-CAM)) was performed using the primary mouse anti-human N-CAM monoclonal antibody (Moc1, diluted 1:1000, DAKO Diagnostics Canada, Inc.) and the ABC-kit. Briefly, the sections were pre-washed for 30 min in a solution of 10% methanol and 3% hydrogen peroxide and blocked in PB containing 0.3% Triton X-100 and 5% normal horse serum (NHS) for 1 hr. The sections were removed and incubated in a 1:1000 solution of monoclonal mouse anti-N-CAM (Moc1) Ab for 16 hr. To visualize Ab binding, 1:250 biotinylated horse anti-mouse IgG Ab (Vector Laboratories, Inc., Burlington, Ontario, Canada) was used, followed by a streptavidin-biotinylated HRP complex kit. The peroxidase was visualized by the addition of DAB.

Staining for the presence of human NSE was performed using the primary mouse anti-NSE monoclonal antibody (1:100; Vector Laboratories Canada, Inc.) and the ABC-kit. The sections were pre-washed for 30 min in a solution of 10% methanol and 3% hydrogen peroxide and blocked in PB containing 0.3% Triton X-100 and 5% NHS for 1 hr. The sections were removed and incubated in a 1:100 solution of mouse monoclonal anti-hNSE Ab for 16 hr. To visualize Ab binding, 1:200 biotinylated horse anti-mouse IgG Ab was used, followed by a streptavidin-biotinylated HRP complex kit and DAB.

All animals that received both intrastriatal and intranigral hNT neuronal grafts had surviving grafts that were strongly immunostained for the presence of both human NSE and human NCAM. Analysis of the transplants by anti-NCAM immunohistochemistry revealed a strong staining of the entire graft area. Darkly NCAM stained cell-like structures could be seen within the graft boundary, and NCAM+ fibers extended beyond the graft-host interface in many of the animals. NSE staining produced a similarly intense pattern, with what appeared to be more darkly stained cells within the graft. NSE+ fibers extended beyond the graft-host interface at the level of the striatum; and in some cases, fibers extended greater than 100 μm into the surrounding host tissue.

Analyses for TH expression are summarized in FIG. 14. No TH+ cells were seen in either the striatum or the SN in animals with hNT neuron grafts (n=16). In 43% of animals with DA neuron grafts (n=4), TH+ cells were readily identified in both the striatum and SN. TH+ neurons were healthy, and their processes extended for variable distances in the host brain. After DA-neuron implants, there were 435.12±323.3 TH+ cells within the striatum and 393.68±204.70 TH+ cells within the SN. In 100% of animals receiving LiCl pre-treated DA-neurons, TH+ cells were observed in the intrastriatal and intranigral grafts. The mean numbers of TH+ cells within the intrastriatal and intranigral grafts were 489.39±18.09 and 319.68±142.08, respectively. There was no significant difference in the number of TH+ neurons between the DA-neuronal and the LiCl pre-treated DA-neuronal grafts (p>0.05). There was no significant difference in the number of TH+ cells between the intrastriatal and intranigral graft locations (p>0.05).

The lack of difference between surviving TH+ neurons in the striatum or SN suggests that the homotopic site (SN) does not influence the phenotype of hNT neurons. This contrasts with a report that hNT neurons differentiated to a dopaminergic phenotype under the influence of mouse caudatoputamen (Miyazono et al. J Comp Neurol 376:603-613, 1996). The lack of significant functional recovery most likely relates to the low number of TH+ neurons, as it has previously been demonstrated that the number of surviving TH+ neurons and fiber outgrowth strongly correlates with the extent of functional recovery. The present study shows that DA-neurons and LiCl-treated DA-neurons survive transplantation in the striatum and SN, integrate into the host, and express TH.

### Example 9

### Post-Cryopreservation Survival and Apoptosis

DA and hNT neurons as well as NT2 precursors were stored at −180° C. prior to use. Freshly thawed cells resuspended in the medium containing DMEM (Gibco, BRL, Grand Island, N.Y.) and 10% fetal bovine serum (Gibco, BRL) were plated on poly-L-lysine coated eight-well chamber slides at a concentration of 100,000 cells/cm². After 24 hr the plating media was switched to DMEM:F12 (Gibco, BRL) containing 0.1% ITS (Sigma), and Gentamicin (50 μg/ml, Sigma). Cultures were either maintained in the second medium for 1 day (1 DIV) or for 5 days (5 DIV), then rinsed in 0.1 M PBS and fixed with 4% paraformaldehyde.

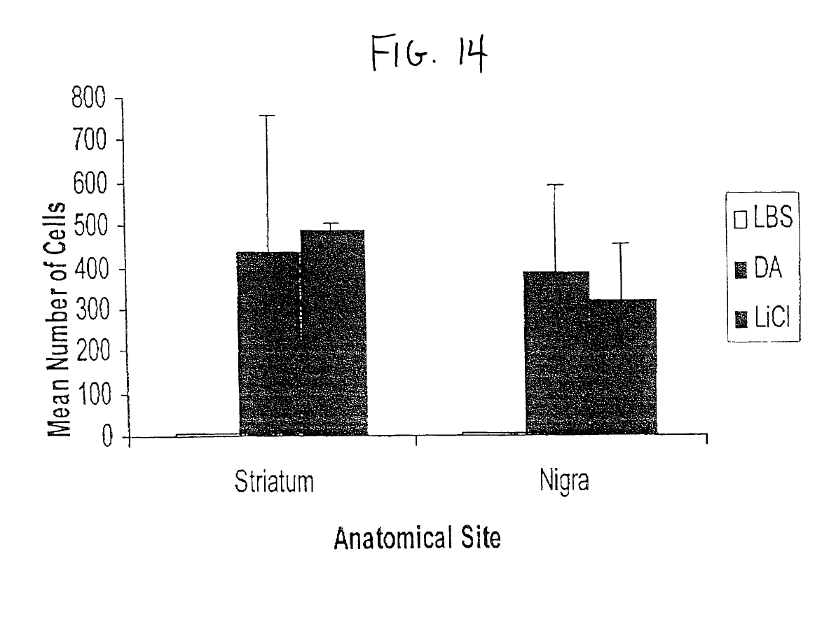

Survival and the morphological appearance of living, non-fixed cultures neurons and precursors were assessed from slides stained with fluorescein diacetate (FDA)-propidium iodide (PI) at 1 DIV or 5 DIV. Only those platings, which revealed the vast majority (85-95%) of healthy FDA+ cells (FIG. 15) evenly distributed throughout a culture dish were selected and processed for apoptosis or TH/DAPI-immunocytochemistry (see below). FIG. 15 shows assessment of cell viability: FIGS. 15A and 15B show NT2 cells, and FIGS. 15C and 15D show DA cells (previously treated with RA for 4 w). Cells cultured for 1 day are shown in FIGS. 15A and 15C, and cells cultured for 5 days are shown in FIGS. 15B and 15D. The vast majority of cells were labeled by FDA, which fluoresced green and was taken up by living cells; the PI fluoresced red and passively accumulated in dead cells (arrow). The bar represents 100 μm.

For the histological determination of apoptosis in vitro, the NeuroTACS™ In Situ Apoptosis Detection Kit (R&D Systems, Minneapolis, Minn.) was used to identify apoptotic nuclei. Freshly fixed cultures were first permeabilized with NeuroPore reagent, and endogenous activity was quenched using H₂O₂. DNA fragmentation in individual apoptotic cells was visualized by detection of biotinylated nucleotides incorporated into the free 3′-hydroxyl residues of these DNA fragments. A streptavidin-conjugated horseradish peroxidase bound to the biotinylated DNA fragments generated brown precipitates in the presence of diaminobenzidine (DAB). Blue counterstaining was used for easier identification of cells. The positive controls were generated by brief treatment of cells with nuclease prior to labeling in order to generate DNA strand breaks in virtually all cells. Negative controls consisted of slides in which terminal deoxynucleotidyl transferase (tdt) was omitted from the reaction mixture. The number of apoptotic nuclei versus total number of cells was determined from three independent culture platings for every RetA exposure and time point (1 DIV and 5 DIV). The number of apoptotic and non-apoptotic cells was counted using a 20× objective placed over two randomly selected non-overlapping sites per well (4 wells/plating, 3 platings in total). Percentages were determined as a ratio of apoptotic cells/total number of cells multiplied by 100. The mean values ±SEM from 0, 3, 4 and 5 weeks RetA exposures at each time point were compared using a one-way analysis of variance followed by Dunnet's post hoc comparisons.

NT2 precursors at 1 DIV plated at the same density as induced cells covered uniformly the surface of the culture dish. NT2 cell bodies were large and flat, occasionally sending out short processes. Numerous NT2 cells were in various stages of mitosis, easily distinguishable after blue counterstaining. The number of apoptotic positive nuclei was very low (3.6%). In 5 DIV cultures, the proliferating precursor cells completely covered the surface of the well forming a tightly packed carpet-like monolayer. Even after longer survival, NT2 precursors revealed low levels of tdt labeling (4.4%). Positive nuclei were usually round with distinct fragmentation (Table 6).

TABLE 6
Apoptosis in NT2, DA (4W) and hNT (5W) Cells
Percentage of tdt-labeled cells

| DIV | NT2 | 4wRA | 5wRA |
|---|---|---|---|
| 1 | 3.6 ± 0.1 (2768) | 12.5 ± 2.0 (3562)* | 12.5 ± 0.5 (2310)* |
| 5 | 4.4 ± 0.3 (1728) | 15.4 ± 1.3 (3572)* | 15.3 ± 1.0 (3284)* |

Data represent the percentages of apoptotic cells vs total number of cells (numbers in parentheses) ±SEM. The asterisk represents a significant difference between non-RetA and RetA-treated cultures in both studied time points; p<0.01.

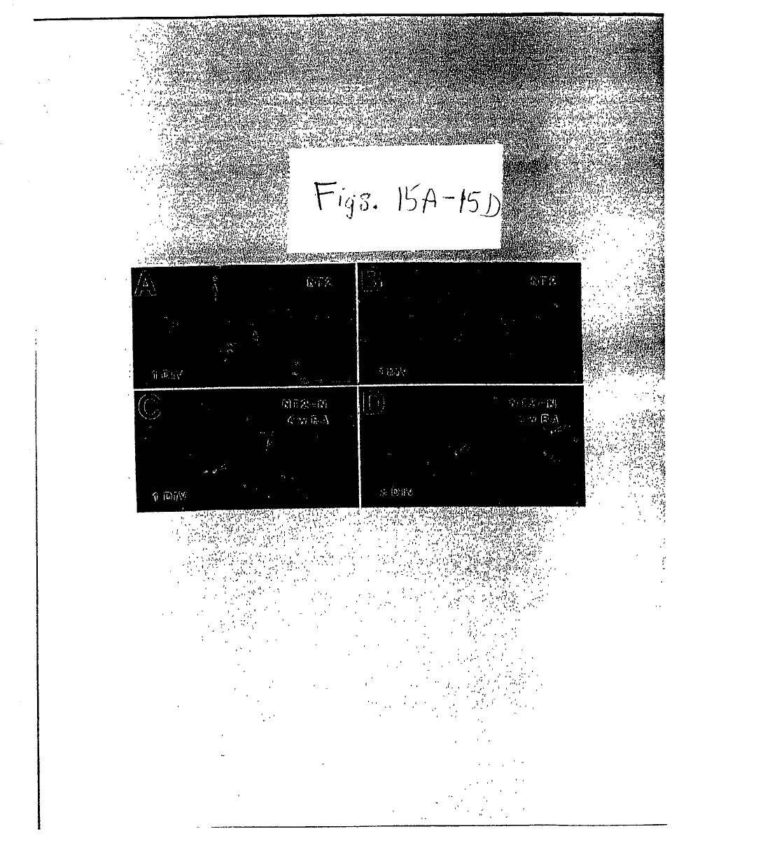

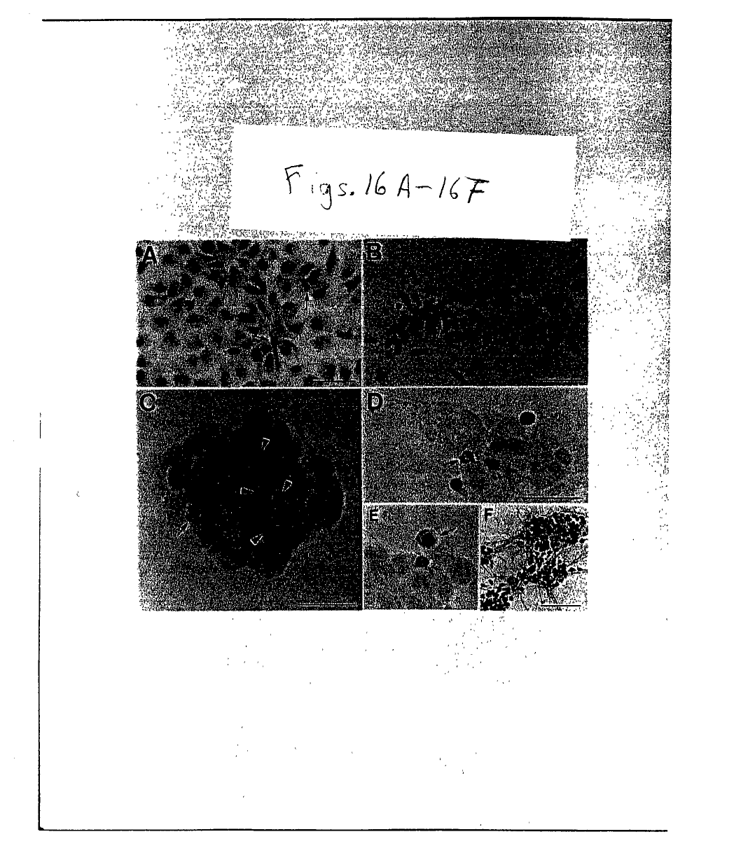

After exposure to RetA, the morphological appearance of differentiated neurons was substantially changed. After 1 DIV individual cells or clusters containing neurons with small usually round bodies and only few, short processes were found. When compared to NT2 undifferentiated cultures, significantly higher numbers of apoptotic-positive nuclei were detected in every RetA-treated group (Table 6). The appearance of DNA condensation varied from small round darkly stained nuclei to those showing a darkly labeled nuclear periphery and weakly labeled center (also called “halo” morphology) or fragmented nuclei broken into several intensely tdt-positive pieces (FIG. 16).

FIGS. 16A-16F show apoptosis in NT2 and induced cells cultured for 5 days. FIG. 16A is a bright-field photomicrograph showing apoptotic nuclei (dark brown, e.g., arrows) in cultured NT2 cells. The bar represents 50 μm. FIG. 16B shows a group of DA neurons (previously treated with RetA for 4 w) with several apoptotic nuclei (arrows). The blue counterstain was used to visualize the cell bodies. The bar represents 50 μm. FIG. 16C shows a clump of DA neurons (4W RetA) showing nuclei with a single (arrowheads) or multiple lobes of condensed chromatin (arrow). The bar represents 25 μm. FIG. 16D is another example from the group of MI cells exposed previously to 3W RetA-treatment. Apoptotic cells indicated by arrows have “halo” morphology. The bar represents 50 μm. FIG. 16E shows that in some dying MI neurons (arrow), cytoplasm is still visible/present. This bar represents 50 μm. FIG. 16F shows the positive control (see above) from the same experimental group as in FIG. 16B. Bar represents 100 μm. Overall the most frequently seen was dark compact nuclear staining. In many instances, the cytoplasm of dying cells was shrunken or substantially reduced.

### Example 10

### Improved Survival of Transplanted DA Neurons Treated with a Lithium Salt

Male Sprague-Dawley rats were assigned to one of the following experimental groups:

a) DA neurons transplanted after 24 hr culture (DA hNT; n=9)

b) DA neurons transplanted after 24 hr culture with 1 mM LiCl (Li+DA hNT; n=9)

c) DA neurons transplanted after 24 hr culture into an animal consuming Li+ in the diet (DA hNT+Li chow; n=9)

d) DA neurons transplanted after 24 hr culture with 1 mM LiCl into an animal consuming Li+ in the diet (Li+ DA hNT+Li chow; n=9)

e) DA neurons maintained in media 2-3 hr prior to transplantation (DA hNT at Thaw; n=9)

f) DA neurons exposed to Li 2-3 hr prior to transplantation only (Li at Thaw; n=13)

This study was conducted under the purview of the University of South Florida IACUC and complied with the NIH guidelines for care and use of animals.

6-Hydroxydopamine Lesioning:

We have shown that the neurotransmitter phenotype of the cells can be influenced both in vitro and in vivo (Zigova et al, Exper Neurol 157: 251-258, 1999; Saporta et al, Brain Res Bull 53: 263-268, 2000). As such, it was felt that for a truly representative test of whether or not TH expression would be maintained against a background of dopamine depletion, the study should be performed in the 6-OHDA lesioned animal. The rats (250-300 g) were anesthetized with Equithesin (3.5 mL/kg), a combination of chloral hydrate and pentobarbital, and positioned in a Kopf stereotaxic frame. With the interaural line as a reference point, a hole was drilled through the skull and the needle positioned so as to lesion the right ascending mesostriatal dopaminergic system (3.6 mm anterior to the interaural line, −1.1 mm lateral and −7.9 mm ventral to the dura with the toothbar set at −2.6 mm). The 6-OHDA (9 μg in 2.5 ml of normal saline with 0.2% ascorbic acid) was injected through a 10 μl Hamilton syringe with a 26 gauge needle at a rate of 1 μl/min. The needle was held in place for an additional 5 min and then slowly withdrawn and the incision closed. Three weeks after this procedure, the lesion was verified by testing for motor asymmetry through administration of apomorphine (prepared in normal saline with 0.2% ascorbic acid; 0.2 mg/kg, sc; Sigma). Only those animals that rotated a minimum of 6 rotations/min in a 30-minute test period were transplanted.

Preparation of DA Neurons:

The DA neurons were obtained from Layton BioScience, Inc. (Sunnyvale, Calif.). They were thawed rapidly at 37° C. and gently transferred to a 15 cc centrifuge tube filled with 10 ml of DMEM with 10% fetal bovine serum (FBS) and 0.1% Gentamicin. The cells were centrifuged at 700 rpm for 7 min, the supernatant discarded and the cells resuspended in 1 mL DMEM/FBS media. Viability and cell number were assessed using the trypan blue dye exclusion method. The viability of the thawed cells ranged from 51% to 66%. The DA neurons were seeded at a density of 100,000 cells/cm² on poly-L-lysine (10 μl/mL; Sigma) coated flask (Nunc) in serum-containing media consisting of DMEM supplemented with 10% FBS, and 50 μg/mL Gentamicin. In some cultures, 1 mM LiCl was added to the plating media. After 24 hours, the cells were lightly trypsinized (0.1%) and washed three times in DMEM:F12. After the final wash, the cells were centrifuged and then resuspended in 1 ml Isolyte S, pH 7.4, a ph-balanced, isotonic multi-electrolyte solution for injection (B Braun McGaw Pharmaceuticals). Viability was determined using trypan blue, and the cell concentration was adjusted to 50,000 cells/μl for transplantation. In those conditions in which the cells were cultured 24 hr, the viability prior to transplantation 96%-100%. Post transplant viability on an aliquot from each group ranged from 36%-93%.

In the second condition, the hNT neurons were thawed at 37° C., transferred to a 15 cc centrifuge tube filled with 10 mL of Isolyte solution either with or without 1 mM LiCl in the media, centrifuged twice and after assessing viability, resuspended in this media at 50,000 cells/μL. Pre-transplant and post-transplant viability of the non-cultured cells ranged from 50%-60%.

Diet:

Half of the animals in each of the groups, which received cultured neurons, were maintained on a diet of regular laboratory chow (Harlan Teklad). The remaining animals consumed a diet that was enriched with 0.24% w/w lithium carbonate (Li₂CO₃). This latter diet was introduced one week prior to transplantation.

Transplantation Protocol:

Animals were anesthetized with Equithesin (0.35 mL/100 g) and placed in a stereotaxic frame. The cells were deposited into two separate sites in the striatum along a single needle tract. The coordinates for the injections were 1.2 mm anterior to bregma, +2.7 mm laterally, and −5.2 mm and −4.7 mm ventral to dura with the toothbar set at zero. Each injection of 2 μl was delivered over 2 minutes. The needle was held in place for an additional 5 minutes before being slowly withdrawn and the incision closed. Two hundred thousand cells total were transplanted.

Tissue Preparation:

One week after transplantation, the rats were sacrificed under deep chloral hydrate (10%) anesthesia. A transcardial perfusion of the brain with 50 ml of 0.1 M PB and then 250 ml 4% paraformaldehyde in 0.1 M PB was performed. The brain was removed, post-fixed, immersed in 20% sucrose and cryopreserved before being sectioned at 30 μm on a cryostat. All sections through the transplant were collected in a series of six wells and stored in Walther's antifreeze until use.

Immunohistochemistry:

To identify the DA neurons within the rat striatum, human specific antibodies were used. Free-floating sections were permeabilized with 10% normal serum, 3% lysine, 0.3% Triton X100 in PBS. The sections were then transferred to primary antibody, human NuMA (1:400, Calbiochem) or human NSE (1:75, Novocastra) in 2% normal serum, 0.3% Triton X-100/PBS and incubated overnight at 4° C. After being rinsed in PBS, the sections were incubated in rat-adsorbed biotinylated horse anti-mouse secondary antibody (Vector Laboratories, Burlingame, Calif.) in 0.3% Triton X100 in PBS for 60 min. The sections were rinsed and then placed in avidin-biotin complex (Standard Elite ABC kit; Vector Laboratories) for 1 hour. The reaction product was visualized with the chromagen, VIP (purple; Vector Laboratories).

Double immunohistochemistry was performed to identify TH-positive DA neurons. The sections were permeabilized as described above and then placed in a cocktail of primary antibodies (the monoclonal antibody NuMA (1:200) and a polyclonal antibody to TH (1:250, Pel-Freez)) for 24 hr at 4° C. The sections were then rinsed in PBS and placed in a cocktail of secondary antibodies for 2 hr. Fluorescein-conjugated goat anti-mouse antibody (1:500, Alexa 488 from Molecular Probes, Eugene, Oreg.) was used to identify NuMA and rhodamine-conjugated goat anti-rabbit (1:800, Alexa 594 Dye from Molecular Probes) to identify TH. The sections were rinsed, mounted and coverslipped with Vectashield (Vector Laboratories).

Image Analysis:

All sections were examined on the Olympus BX-60 microscope and all images captured using the Optronics MagnaFire digital camera. To determine cell survival, the NuMA-positive cells were quantified semi-automatically using the Image-Pro II image analysis system. NuMA-positive cells were manually highlighted in sections taken at 180 μm intervals through the graft site in the striatum and then counted by the computer program. In those sections in which double immunohistochemistry was performed, NuMA-positive cells visualized with an FITC conjugated secondary antibody, TH-positive cell bodies (rhodamine) and cells expressing both immunomarkers were counted. In order to be considered double-labeled, the labeled nucleus had to be clearly surrounded by a TH-positive (rhodamine) cell body; if this could not be unequivocally demonstrated, then a cell was not considered to be double-labeled. Neural process growth was also examined from the NSE staining.

Statistical Analysis:

All results were reported as a mean ±SEM. When the data were analyzed with homogeneity of variance tests, it was determined that the data were not normally distributed. Therefore, all results were analyzed with nonparametric statistics. The Kruskal-Wallis test of multiple independent groups was performed first. If significant differences were observed in this analysis, post-hoc testing was performed using the Mann-Whitney U test.

### RESULTS

Cell Survival:

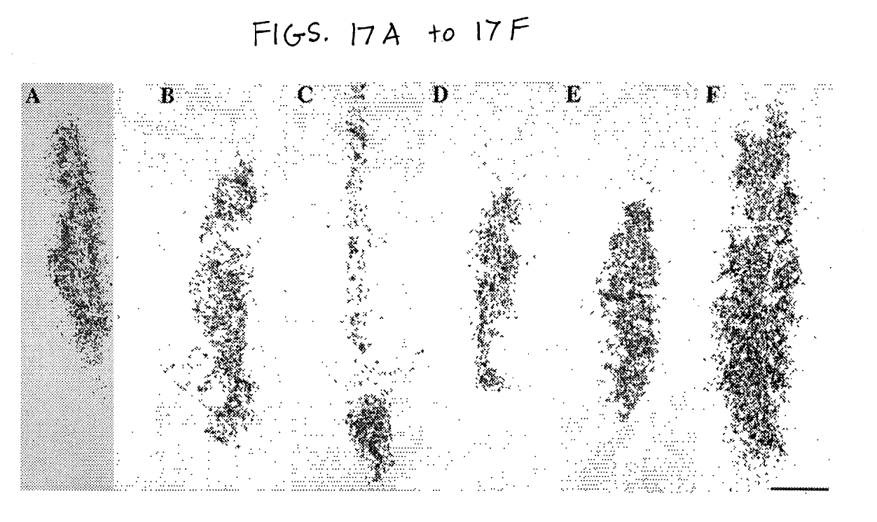

The effect of lithium exposure on cell survival within the graft depended on the specific treatment strategy. See FIGS. 17A-17F, which are photomicrographs of DA neuron grafts in the striatum of hemiparkinsonian rats. FIG. 17A shows DA neurons precultured for 24 hr; FIG. 17B shows DA neurons +Li chow; FIG. 17C shows Li+ DA neurons; FIG. 17D shows Li+ DA neurons +Li chow; FIG. 17E shows DA neurons at Thaw; and FIG. 17F shows Li at Thaw. The scale bar represents 200 μm.

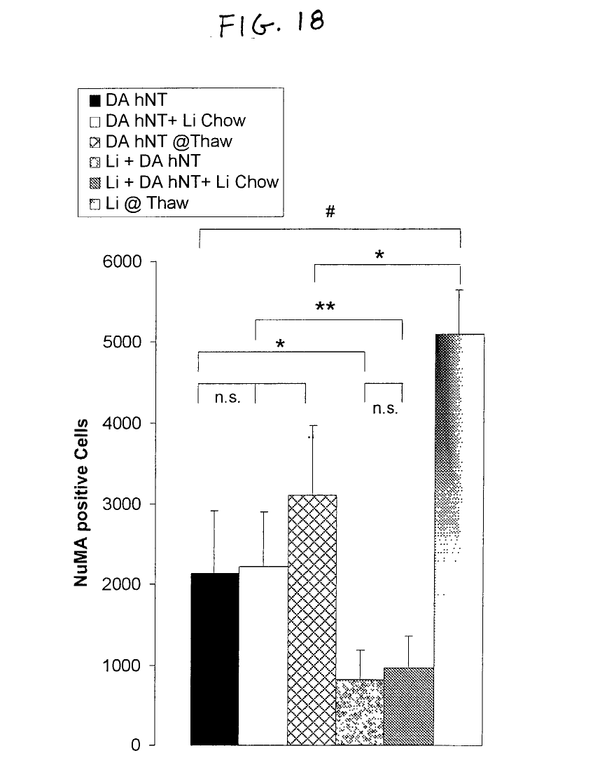

Examination of sections that had been immunostained for NuMA to identify the number of surviving DA neurons revealed many positively labeled cells in all groups. As can be seen in FIG. 17, however, there were more neurons in some groups than others. When the number of NuMA positive cells per group was determined, this qualitative observation was confirmed by the quantitation of DA neurons, summarized in FIG. 18. A single * means p=0.02; two ** means p=0.05 and # means p=0.005. Analysis with the Kruskal-Wallis test showed that there were significant differences between the experimental groups (χ²=21.69, df=5, p=0.0006). The post-hoc testing with the Mann Whitney test, however, failed to demonstrate any significant differences in cell survival between the DA neurons group and the DA neurons +Li chow group (z=0, p=0.5), nor between the Li+ DA neurons group and the Li+ DA neurons +Li chow group (z=-0.6944, p=0.24). However, there were significantly more NuMA positive cells in the DA neurons and DA neurons +Li chow groups compared to animals in the Li+ DA neurons (z=−2.08, p=0.02) or the Li+ DA neurons +Li chow groups (z=−1.61, p=0.05). Perhaps the most interesting results, though, were observed when we compared the Li at Thaw group to DA neurons at Thaw and the DA neurons groups; there were significantly more NuMA+ cells in the Li at Thaw group than in either the DA neurons at Thaw (z=−1.97, p=0.02) or the DA neurons group (z=−2.55, p=0.005).

Tyrosine Hydroxylase Expression:

Using double immunofluorescence techniques with NuMA labeling to identify the neurons and antibodies to TH, we examined whether these cells remained TH+ one week after implantation. The average number of surviving cells and average number of surviving cells that expressed TH are presented in Table 7.

TABLE 7
Tyrosine Hydroxylase Expression in Transplanted Neurons

| | | TH+ DA hNT | % of Double- |
|---|---|---|---|
| Group | NuMA | Neurons | Labeled Cells |
| DA neurons | 772.2 | 75.6 | 9.79 |
| DA neurons + Li Chow | 280 | 49.8 | 17.79 |
| DA neurons at Thaw | 267 | 69.3 | 25.96 |
| Li + DA neurons | 253 | 28 | 11.07 |
| Li + DA neurons + | 402 | 78.7 | 19.58 |
| Li Chow | | | |
| Li at Thaw | 423.7 | 261 | 61.60 |

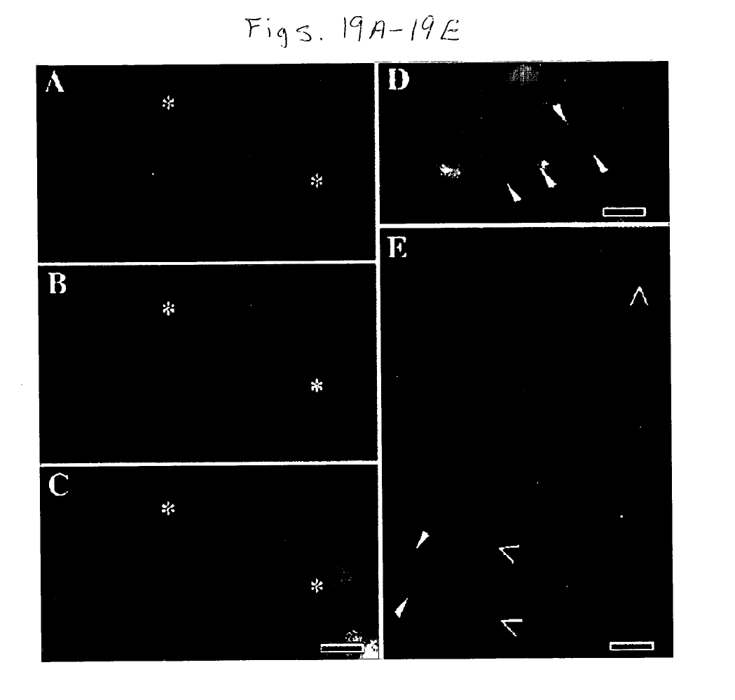

One week after implantation, DA neurons from all of the experimental conditions expressed TH as well. FIG. 19A shows NuMA-labeled DA neurons in the striatum. FIG. 19B shows TH-positive cells in the striatum. FIG. 19C shows TH-positive, NuMA-labeled DA neurons. The asterisks in FIGS. 19A-19C indicate the same cells within the graft. FIGS. 19D and 19E enable a striking observation with the double immunofluorescent labeling of TH-positive DA neurons which was the presence of rarely seen TH-positive fibers extending from the cell bodies (white arrowheads) within the graft. These were observed in all groups. The scale bars represent 10 μm. The TH-positive cells in the host striatum were always double labeled with NuMA (FIGS. 19A-C). Many double labeled cells exhibited well developed TH-positive neuronal processes (black arrowheads) (FIGS. 19D-E).

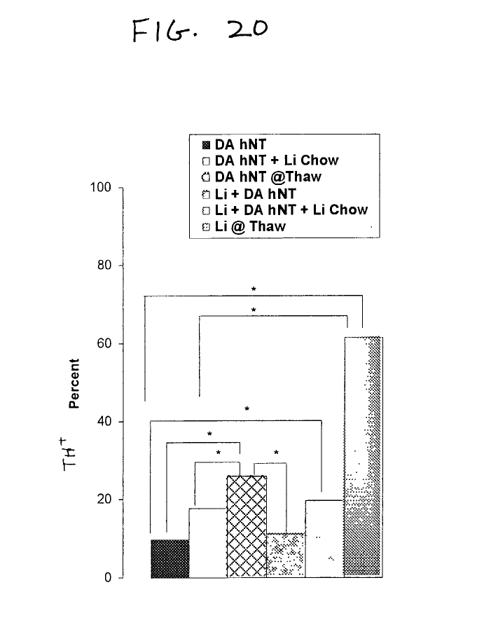

When we examined whether there was a similar percentage of TH expression in the grafts across conditions, we saw significant differences between the experimental groups (χ²=12.89, df=5, p=0.02). FIG. 20 shows the percentage of grafted DA cells that expressed TH and the significant differences (* p=0.01). There were significantly more TH-positive DA neurons in the Li+ DA neurons +Li chow and the DA neurons at Thaw and the Li at Thaw groups compared to the animals in the DA neurons and the Li+ DA neurons group (p=0.05 to 0.01). Similarly, the DA neurons at Thaw and the Li at Thaw groups had significantly more TH-positive cells than the DA neurons +Li group (p=0.05 to 0.01). The most TH-positive cells were observed in the Li at thaw group (62%).

Fiber Outgrowth from the Transplanted Cells:

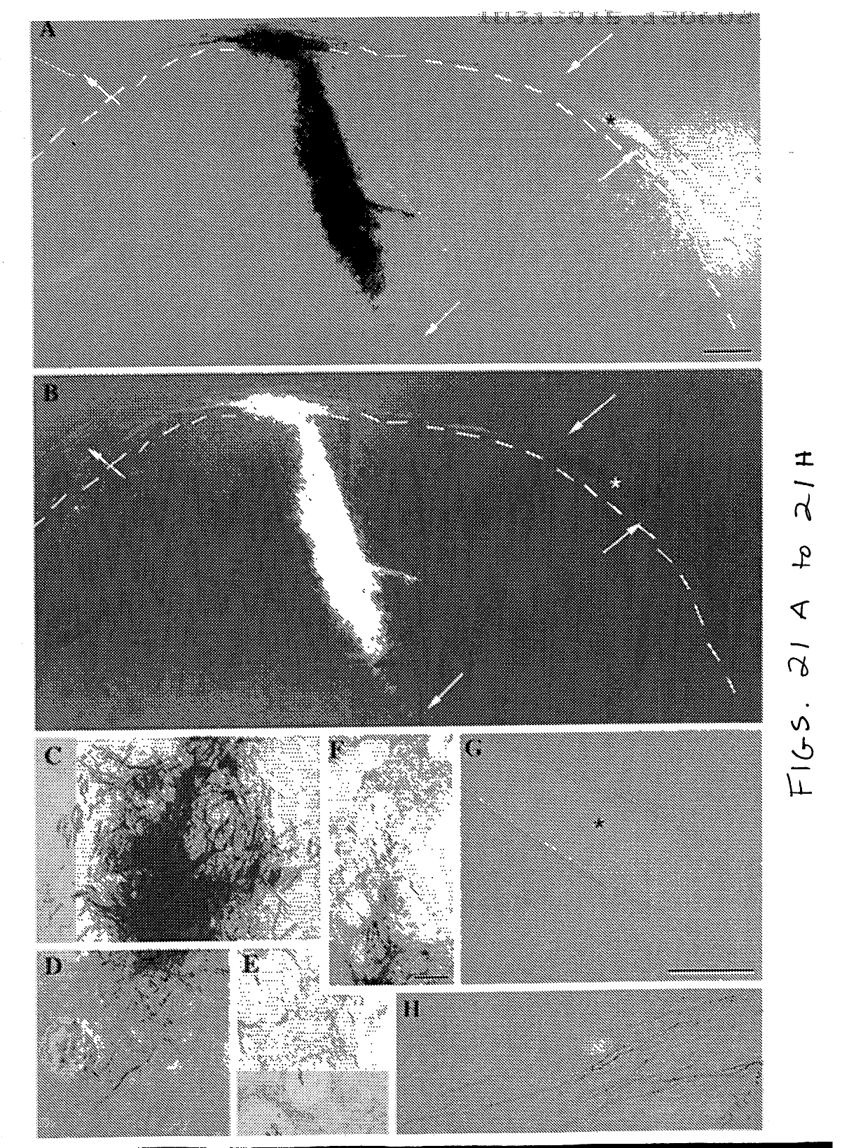

To determine whether Li exposure affected fiber outgrowth from the DA neurons, we immunostained sections through the graft with an antibody to human NSE. FIGS. 21A-21H display fiber outgrowth from the DA neuron grafts. After one week in situ the DA neurons were developing extensive neuritic processes that extended throughout the host striatum. FIG. 21A shows NSE staining of a graft in the striatum. Notice that the most dorsal part of the graft transects the subcortical white matter. The most extensive neuritic outgrowth from the graft occurred in this region. Fibers from these cells were found to extend up to 2 mm from the cell body. The scale bar is 200 μm. FIG. 21B is an image produced by converting the montage in FIG. 21A to a negative photographic image using Photoshop (Adobe Systems), which delineates the full extent of fiber outgrowth from the graft. The fibers coursing toward the corpus callosum, in the lateral subcortical white matter as well as fibers leaving the ventral portion of the graft to enter host striatum are more clearly visible (arrows). In addition, the patch matrix architecture of the striatum is more apparent in this image. FIG. 21C illustrates that within the graft the labeled fibers were so dense, that cell bodies were only visible along the margins of the graft. FIGS. 21D-21F show that within the striatum, processes that exited the graft were mainly found traveling through the fascicles. FIG. 21G is a higher magnification view of fibers in the subcortical white matter shown in FIGS. 21A and 21B (asterisk). FIG. 21H shows some fibers crossing the midline of the corpus callosum. The scale bars in FIGS. 21C-21H represent 50 μm.

As can be seen in FIGS. 21A and 21B, there was extensive labeling of fibers both within and around the graft.

### Discussion

Treating the DA neurons with lithium prior to transplantation can be either beneficial or harmful to cell survival, depending on the protocol used; the timing and duration of exposure to lithium appear to be important factors in the outcome of the treatment. A brief exposure (2-3 hr) just prior to transplantation doubled survival of the DA neurons after transplantation, while a 24 hr exposure to the same concentration in culture resulted in a 65% decrease in cell survival compared to the cultured DA neurons and an 83% decrease compared to the briefly exposed group (Li at Thaw). While it is well documented that lithium is neuroprotective (Centeno and Mora, 1998, NeuroReport 9:4199-4203; Grignon et al., 1996, Eur J Pharmacol 315:111-4; Nonaka et al., 1998, Proc Natl Acad Sci USA, 95:2642-7; Wei et al., 2000, Eur J Pharmacol 392:117-23), it has also been shown that chronic lithium treatment can be associated with a reduction in cell viability (Becker and Tyobeka, 1990, Leuk Res, 14:879-84; Hasgekar et al., 1996, Cel Biol Int, 20:781-6). This is most easily explained by the observation of vastly different concentrations of lithium administration. Those studies that found a neuroprotective effect used lithium concentrations within the therapeutic range for treatment of mood disorders (0.1 to 1.0 mM) while those that found toxicity used concentrations ranging from 2.5 mM to 10 mM. This does not explain why altering the duration of exposure to lithium while maintaining the dose of lithium (1.0 mM) would so drastically alter cell survival. One possible explanation may be that there is a time course of action that is critical to the observed effect. However, when the lithium effect on neuronal populations has been examined over time, chronic exposure has usually been required before the optimal outcome is observed (Nonaka et al., 1998, Proc Natl Acad Sci USA, 95:2642-7). Further, there was no increase in apoptotic cell death over time in tissue culture studies where lithium was present for 10 days (Zigova et al., 2001, Dev Brain Res, 127:63-70). The observed decrease in cell survival in the grafts of animals that received DA neurons that were cultured for 24 hours prior to transplantation may have more to do with the maturity of the lithium-treated cells after 24 hours in culture, making it difficult to lift the cells from the culture plate without injuring neuritic processes. This argues directly against the observations of D'Mello et al (1994), in which lithium-induced apoptosis in immature cerebellar granule cells in vitro but promoted cell survival in mature granule cells. The only other published study that may indirectly address this issue is a recent study that reported better graft survival of the DA neurons when lithium was added to the replate media during the production of the cells prior to the cells being harvested and cryopreserved for later use (Baker et al., 2000, Exp Neurol, 16:350-60).

A second important observation is that briefly exposing the DA neurons to lithium prior to transplantation may help to stabilize TH expression in the grafts. In culture, 60% of the DA neuron population is dopaminergic (Zigova et al., 2000, Dev Brain Res, 122:87-90); further, in hNT neurons that were not optimized for TH expression but were exposed to LiCl to increase the number of cells that express TH, subsequent removal of the Li does not decrease TH expression (Zigova et al., 1999, Exp Neurol, 157:251-258). The expression of the TH gene is driven by a promoter containing an activator protein 1 (AP-1) binding site (Chen et al., 1997, Neuropsychopharmacology, 16:238-245); lithium apparently modulates gene expression through this transcription factor pathway, indirectly increasing AP-1 binding (Ozaki and Chuang, 1997, Neurochem, 69:2336-2344; Yuan et al., 2001, J Biol Chem, 276:31674-83) and both c-fos and c-jun mRNA and protein expression (Ozaki and Chuang, 1997). Similarly, lithium has also been shown to modulate gene expression through cyclic AMP-responsive element (CRE) binding (Ozaki and Chuang, 1997; Unlap and Jope, 1997, Neuropsychopharmacology, 17:12-7). In the animals in the Li at Thaw group, TH was still expressed in 60% of the DA neurons after one week in vivo. In all other groups, TH expression had declined to between 11 and 27% of the surviving cells in the graft within the same time period. At this time it is unclear whether this decrease in expression is part of a down-regulation of expression or is a temporary phenomenon related to cell maturation or surgical trauma. Certainly, it has been shown that neuronal differentiation and TH expression of NT2 precursors of the DA neurons may increase with time after transplantation (Miyazono et al., 1996, J Comp Neurol, 376:602-613).

Cell morphology and integration into the host were also of interest. TH-positive hNT neurons treated with 1 mM LiCl in culture were larger and had longer neurites, which exhibited more branching (Zigova et al., 1999, Exp Neurol, 157:251-258). Others have also found that lithium promotes extension (Garcia-Perez et al., 1999, Neurosci Res, 57:261-70; Hasgekar et al., 1996, Cell Biol Int, 20:781-6) or branching of neurites in a dose dependent manner (Lucas and Goold, 1998, J Cell Sci, 111:1351-1361; Lucas and Salinas, 1997, Dev Biol, 192:31-44). It has been hypothesized that these changes in neurite extension occur because lithium reduces phosphorylation of tau, thereby enhancing tau binding to microtubules and promoting microtubule assembly within the neurite (Hong et al., 1997, J Biol Chem, 272:25326-32). Growth cone area, perimeter and length increase at a variety of Li concentrations (Lucas and Goold, 1998, J Cell Sci, 111, 1351-1361). However, at higher concentrations (25 mM), neurite extension decreases (Hollander and Bennett, 1991, J Neurosci Res, 28:332-42). When an extracellular matrix is provided, neurite outgrowth can occur even at the moderately high concentration of 10 mM Li (Lamoureux et al., 1990, Cell Biol, 110:71-9).

Extensive neuritic outgrowth from the transplants was visible with staining for NSE in all groups. Because many fibers were cut in the sectioning process and a truly representative estimate of fiber length may not have been possible, it is difficult to irrefutably determine differences between the groups. An interesting observation in all groups was that the fibers appeared to be attracted to white matter tracts, coursing through the fascicles of the striatum and the subcortical white matter. This is consistent with observations made when hNT neurons were transplanted into the spinal cord (Hartley et al., 1999, J Comp Neurol, 415:404-418). This propensity may speak directly to the models or diseases in which these cells may be useful as treatments as well as modifying existing protocols for treatment. For example, reconstruction of the ascending mesostriatal system may include grafts into the medial forebrain bundle instead of (or in addition to) the striatum or substantia nigra.

The combination of improved survival and higher TH expression after brief lithium exposure is especially beneficial in transplantation of cells used to treat a disease such as Parkinson's disease.

The foregoing description and examples are intended only to illustrate, not to limit, the disclosed invention.

What is claimed is:

1. A method of improving the survival of neuronal cells, the method comprising
a. obtaining a frozen cellular composition comprising neuronal cells;
b. thawing the cellular composition; and
c. contacting the cellular composition with a balanced electrolyte solution comprising a lithium salt.

2. The method of claim 1 wherein the lithium salt concentration is greater than about 0.25 mM and less than about 5 mM.

3. The method of claim 1 wherein the lithium salt concentration is in the range of about 0.5 mM to about 3 mM.

4. The method of claim 1 wherein the lithium salt concentration is in the range of about 0.75 mM to about 2 mM.

5. The method of claim 1 wherein the lithium salt concentration is about 1 mM.

6. The method of claim 1 wherein the lithium salt is lithium chloride.

7. The method of claim 1 further comprising the step of centrifuging the thawed cellular composition and removing a resulting supernatant.

8. The method of claim 1 further comprising the step of
d. assessing viability of a portion of the neuronal cells in a balanced electrolyte solution.

9. A kit comprising
a. a container with a cellular composition comprising committed neuronal cells; and
b. a container of a diluent comprising a balanced electrolyte solution and a lithium salt.

10. The kit of claim 9 wherein the lithium salt concentration is greater than about 0.25 mM and less than about 5 mM.

11. The kit of claim 9 wherein the lithium salt concentration is about 0.5 mM to about 3 mM.

12. The kit of claim 9 wherein the lithium salt concentration is between about 0.75 mM and about 2 mM.

13. The kit of claim 9 wherein the lithium salt concentration is about 1 mM.

14. A method of increasing the numbers of dopaminergic cells in a cellular composition comprising neuronal cells, the method comprising
a. providing a cellular composition comprising neuronal cells; and
b. contacting the composition with a balanced electrolyte solution comprising a lithium salt for less than about 4 hours,
thereby increasing the numbers of dopaminergic cells in the composition.

15. The method of claim 14 wherein the lithium salt concentration is less than about 0.25 mM and greater than about 5 mM.

16. The method of claim 14 wherein the lithium salt concentration is in the range of about 0.5 mM to about 3 mM.

17. The method of claim 14 wherein the lithium salt concentration is in the range of about 0.75 mM to about 2 mM.

18. The method of claim 14 wherein the lithium salt is lithium chloride.

19. The method of claim 14 further comprising the step of centrifuging the cellular composition and removing a resulting supernatant.

20. The method of claim 14 further comprising the step of
d. assessing viability of a portion of the neuronal cells in the balanced electrolyte solution.
